(19) United States
(12) Patent Application Publication
(10) Pub. No.: US 2025/0000337 A1
(43) Pub. Date: Jan. 2, 2025

(54) ROBOTIC CLEANING DEVICES AND SYSTEMS FOR SURGICAL INSTRUMENTS, AND METHODS THEREOF

(71) Applicant: Bayou Surgical, Inc., Houston, TX (US)

(72) Inventors: Ashok GOWDA, Bellaire, TX (US); Kimberly NAJJAR, Houston, TX (US); Rebecca SMITH, Houston, TX (US); Megan GREENWOOD, Houston, TX (US); Abdul UMARU, Houston, TX (US); Jorge SALAZAR, Houston, TX (US); William E. COHN, Bellaire, TX (US); Scott SLOSS, Houston, TX (US)

(21) Appl. No.: 18/885,494

(22) Filed: Sep. 13, 2024

Related U.S. Application Data
(63) Continuation of application No. PCT/US2023/064479, filed on Mar. 15, 2023.
(60) Provisional application No. 63/320,166, filed on Mar. 15, 2022.

(51) Int. Cl. A61B 1/00; A61B 1/12

(52) U.S. Cl. CPC A61B 1/00142; A61B 1/122

(57) ABSTRACT

Embodiments described herein relate to systems, devices, and methods of cleaning endoscopes or other instruments during surgical procedures, which can be used with a robotic system.

### CROSS-REFERENCE TO RELATED APPLICATIONS

This application is a continuation of International Application No. PCT/US2023/064479, filed Mar. 15, 2023, titled “ROBOTIC CLEANING DEVICES AND SYSTEMS FOR SURGICAL INSTRUMENTS, AND METHODS THEREOF,” which claims priority to U.S. Provisional Patent Application No. 63/320,166, filed Mar. 15, 2022, titled “ROBOTIC CLEANING DEVICES AND SYSTEMS FOR SURGICAL INSTRUMENTS,” the disclosures of which are hereby incorporated by reference in their entirety.

### TECHNICAL FIELD

The present disclosure relates generally to systems, devices, and methods for cleaning instruments. More specifically, the present disclosure relates to cleaning of imaging devices that are positioned within a subject's body during robot-assisted surgical procedures.

### BACKGROUND

Trocars are medical devices that are used during minimally-invasive surgical procedures such as laparoscopy, endoscopy, etc. During these surgical procedures, a trocar can be inserted into a subject's anatomy (e.g., body lumens or cavities). After insertion, the trocar becomes a vehicle for introducing surgical instruments and imaging devices (e.g., cameras) that aid with the surgical procedure. For example, an imaging device (e.g., cameras such as a laparoscope, an endoscope, or other fiberscopes) can be inserted into a subject's anatomy (e.g., interiors of body lumens or cavities) through the trocar. These imaging devices can be used to visualize the internal organs of a subject to make diagnosis and/or to gain access to these internal organs for treatment. In addition, trocars can allow for venting of gas or fluid from internal organs within the subject's body.

Given that the imaging devices are inserted into the subject's body, the tip of these imaging devices (e.g., tip of the laparoscope, endoscope, or other fiberscopes that is inserted into the subject's body) can become obscured, due to smears, residue, debris, condensation, and/or other types of obstructing material. For instance, fluid or gas within the subject's internal organ can obscure the lens of the imaging device. Therefore, the tip of the imaging devices need to be cleaned during the medical procedure.

Conventionally, to clean the imaging devices, a surgeon may retract the imaging device from the subject's body, clean the imaging device, and then re-insert the imaging device through the trocar. But, such removal and re-insertion can add to the time of surgery and increase the risk of infection (e.g., due to a sterile device being removed from a body cavity).

Recently, robot-assisted surgeries have been widely adopted for minimally-invasive surgical procedures. Robot-assisted surgeries allow surgeons to perform minimally-invasive surgical procedures with greater precision, flexibility, and control. During a robot-assisted surgical procedure, the surgeon may make tiny incisions on the subject's body. The surgeon may control a robot thereafter which in turn manipulates the trocar, the surgical instruments, and the imaging devices with precision. However, as discussed above, the tip of the imaging devices can become obscured during the surgical procedure. To remove the imaging device, a human may need to enter the surgical room and manually remove the imaging device from the robot, clean it, and then reattach the imaging device to the robot. Such procedures can be burdensome, require the need for human operators, and increase the risk of infection. Moreover, such steps can delay the surgical procedure and potentially impact the precision of the robot-assisted surgery.

Accordingly, there exists a need for an improved, precise, and automated way to clean the end of the imaging devices during surgical procedures such as robot-assisted surgical procedures.

### SUMMARY

Embodiments described herein relate to a trocar designed to clean the end of an imaging device, such as, for example, an endoscope. The trocar and the endoscope can be positioned within a body cavity. The trocar can use quantities of liquid and/or gas to clean the end of the endoscope. In some embodiments, the imaging device can be coupled to a robotic device, which can be configured to manipulate the imaging device. For example, the robotic device can be configured to move the imaging device for viewing of the body cavity and/or cleaning of the distal end of the imaging device. In some embodiments, the robotic device can be configured to retract the imaging device to position it for cleaning, e.g., via the trocar. In some embodiments, the robotic device can be programmed to position the imaging device in a suitable location for cleaning by a trocar via a calibration process.

In some embodiments, an apparatus includes: a shaft having a distal end that is disposable within a subject's anatomy, the shaft including an ejection port disposed near the distal end of the shaft, the ejection port configured to eject a volume of liquid or gas into the channel to clean a distal end of the instrument; a hub coupled to a proximal end of the shaft, the hub and the shaft collectively defining a channel for receiving a trocar of a robotic system; a sensor disposed near the ejection port, the sensor configured to detect when a distal end of an instrument disposed within a channel of the trocar is disposed near the ejection port; and a controller operatively coupled to the sensor, the controller configured to: receive, from the sensor, a signal indicative of the distal end of the instrument being disposed near the ejection port; and in response to receiving the signal from the sensor, activate a wash sequence by triggering the ejection of the volume of liquid or gas.

In some embodiments, a system includes: a shaft defining a channel for receiving an instrument, the shaft having a distal end that is disposable within a subject's anatomy, the shaft including an ejection port configured to eject a volume of liquid or gas into the channel to clean a distal end of the instrument; a fluid delivery system coupled to the shaft and configured to control the ejection of the volume of liquid or gas; a robotic system coupleable to the instrument and configured to retract the instrument to a predetermined position; and a processor operatively coupled to the robotic system and the fluid delivery system, the processor configured to control the fluid delivery system to cause the ejection of the volume of liquid or gas in response to the instrument being retracted to the predetermined position.

In some embodiments, a method includes: positioning a trocar within a shaft of a cleaning system such that a distal end of the trocar is disposed within a channel of the shaft, the shaft having a proximal end that is coupled to a hub and a distal end that is disposed within a body cavity; securing a proximal end of the trocar to the hub after the trocar has been positioned within the shaft; inserting an endoscope into a channel of the trocar such that a distal end of the endoscope extends distally from the distal end of the trocar, into the channel of the shaft, and then into the body cavity; retracting the endoscope such that the distal end of the endoscope is within the channel of the shaft and near an ejection port within the shaft to be cleaned by a volume of liquid and gas that is ejected by the ejection port into the channel.

In some embodiments, a method includes: detecting that a distal end of an endoscope is near an ejection port of a first trocar disposed in a body cavity, the endoscope being supported within a channel of a second trocar that is disposed within a channel of the first trocar; in response to the detecting, activating a delivery of pressurized gas to propel and deliver a predetermined volume of liquid to the first trocar; and delivering, via the ejection port, the pressurized gas and the predetermined volume of liquid into the channel of the first trocar to clean the distal end of the endoscope.

### BRIEF DESCRIPTION OF THE DRAWINGS

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally similar and/or structurally similar elements).

FIG. 1 is a block diagram of a system for cleaning an imaging device in an interior portion of a subject's anatomy (e.g., body lumen or cavity), including a robotic system, according to an embodiment.

FIG. 2 is a block diagram showing a more detailed view of a fluid delivery system for cleaning an imaging device disposed in a subject's anatomy (e.g., body lumen or cavity), according to an embodiment.

FIG. 3 is a block diagram showing a more detailed view of connections between a fluid delivery system and a trocar of a cleaning system, according to an embodiment.

FIG. 4 is a schematic diagram of a robotic system for cleaning imaging device, according to an embodiment.

FIG. 5 is a block diagram of a trocar of a cleaning system, according to an embodiment.

FIG. 6 is a block diagram of an obturator that can be used with a trocar of a cleaning system, according to an embodiment.

FIG. 7A is an illustration of a trocar and an obturator of a cleaning system, and various components thereof, according to an embodiment.

FIG. 7B provides a detailed view of a distal end of the trocar of FIG. 7A.

FIG. 8A is an illustration of a trocar of a cleaning system and an imaging device (e.g., scope) coupled to a robotic system, according to an embodiment.

FIG. 8B depicts a guide used for calibrating the position and/or orientation of an imaging device relative to a trocar of a cleaning system, according to an embodiment.

FIG. 8C depicts the relative positioning of a distal end of an imaging device and a distal end of a trocar of a cleaning system, according to an embodiment.

FIGS. 9A and 9B depict a guide used for calibrating the position and/or orientation of an imaging device relative to a trocar of a cleaning system, according to an embodiment.

FIGS. 10A and 10B are illustrations of external sensing components configured to detect a position and/or orientation of an imaging device (e.g., scope), according to an embodiment.

FIG. 11 illustrates a method associated with operating a cleaning system, according to an embodiment.

FIGS. 12A and 12B illustrate a cleaning system in which a first trocar of a robotic system is nested within a second trocar of a cleaning system, according to embodiments.

FIG. 13 illustrates a cleaning system in which a first trocar of a cleaning system is nested within a second trocar of a robotic system, according to embodiments.

### DETAILED DESCRIPTION

Systems, devices, and methods for cleaning imaging devices (e.g., cameras such as endoscopes, laparoscopes, and other fiberscopes) during a surgical procedure are descried herein. More specifically, systems, devices, and methods for cleaning imaging devices in an automated and precise manner during a surgical procedure (e.g., robot-assisted surgical procedure) are described herein.

The technology described herein can include a robotic system to automatically clean imaging devices during a surgical procedure. In some embodiments, the robotic system can aid surgeons during a surgical procedure by performing at least a portion of the surgical procedure. In some embodiments, the robotic system can be configured to determine that an imaging device inserted into a subject's (e.g., patient's) body during the surgical procedure requires cleaning. For example, the robotic system can analyze image data from the imaging device and determine that the imaging device requires cleaning based on the analysis. Additionally or alternatively, the robotic system can be configured to receive an input from a user (e.g., surgeon, operator, etc.) indicating and/or instructing the robotic system to clean the imaging device. Additionally or alternatively, the robotic system can be configured to clean the imaging device at predetermined time points and/or predetermined time intervals during the surgical procedure.

In some embodiments, in response to determining that the imaging device requires cleaning, the robotic system can be configured to retract a tip of the imaging device from the subject's body. The robotic system can control a cleaning system to clean the imaging device. For example, the robotic system can be configured to retract the imaging device to a position and/or orientation at which the imaging device can be cleaned. In some embodiments, the robotic system can be coupled to or include a fluid delivery system that can use gas to propel small, controlled amounts of a liquid into a lumen of a trocar to clean the imaging device. After the imaging device has been cleaned, the robotic system can be configured to re-position and/or re-orient the imaging device (e.g., in an automatic manner) such that the surgical procedure can continue in a seamless manner without adversely affecting the surgical procedure.

In some embodiments, the robotic system can be configured clean the imaging device based on a cleaning type and/or an intensity of cleaning. For example, the robotic system can analyze image data from the imaging device and determine a type of cleaning that may be required and/or an intensity of cleaning that may be required. For instance, if the robotic system detects the presence of a large quantity of obstructing material on the imaging device based on the image data, the robotic system can automatically select a high intensity cleaning cycle to thoroughly clean the imaging device. Additionally or alternatively, a user can select the type of cleaning and/or the intensity of cleaning that may be required for the imaging device and can instruct the robotic system to clean the imaging device accordingly. In some embodiments, one or more sensors that are internal to the trocar and/or external to the trocar can be used to sense the position and orientation of the imaging device to automate the cleaning process.

Examples of endoscope cleaning systems are described in U.S. Patent Publication No. 2019/0125176, filed Oct. 18, 2018, and titled, “Trocars,” U.S. Patent Publication No. 2021/0127963, filed Nov. 21, 2019, and titled “Intraoperative Endoscope Cleaning System,” and U.S. Patent Publication No. 2021/0127964, filed Nov. 21, 2019, and titled “Intraoperative Endoscope Cleaning System,” the disclosure of each of which is hereby incorporated by reference in its entirety. Systems, devices, and methods described herein improve on such cleaning systems, e.g., by automating certain cleaning operations and positioning of instruments.

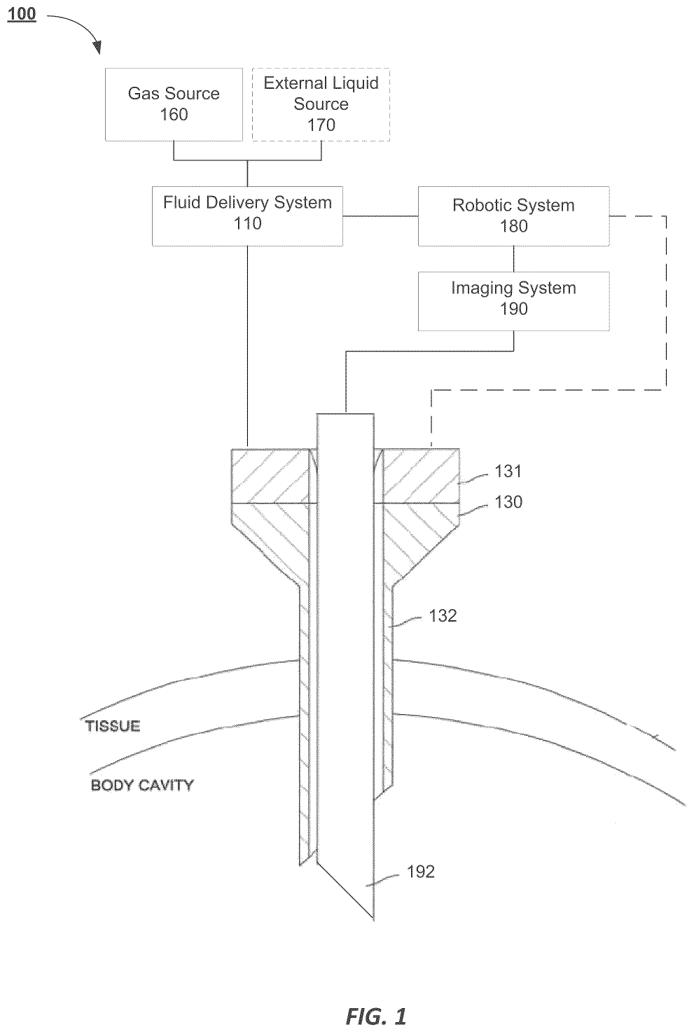

FIG. 1 is a block diagram of a system 100 for cleaning an imaging device in an interior portion of a subject's anatomy (e.g., body lumen or cavity), including a robotic system 180, according to an embodiment. During a minimally-invasive surgical procedure, incision(s) can be made on a body of a subject. A trocar 130 can be inserted into the body of the subject via these incision(s) such that the trocar 130 can be disposed within a body cavity that lies below a layer of tissue. The trocar 130 can be operably coupled to an imaging system 190. In some embodiments, the imaging system 190 can include an imaging device (e.g., endoscope, laparoscope, fiberscope, etc.), such as imaging device 192. Additionally or alternatively, the imaging system 190 can be coupled to the imaging device 192. A robotic system 180 can be coupled (e.g., communicably coupled and/or operably coupled) to the imaging system 190 and the fluid delivery system 110. The fluid delivery system 110 can be fluidly coupled to the gas source 160 and optionally coupled to the external liquid source 170. In some embodiments, the robotic system 180 can be optionally coupled to the trocar 130.

The trocar 130 is a surgical instrument that can be placed within a subject. For example, the trocar 130 can be placed into a body lumen and/or a cavity of the subject. Some non-limiting examples of body cavity can include an abdomen, a thoracic cavity, a chest cavity, gallbladder, bladder, kidney, lung, a combination thereof, and/or the like. A trocar 130 can include (1) a trocar hub 131 to provide a handle for placement of the trocar 130 and/or an anchor port for securing the trocar 130 and (2) a trocar shaft 132 that extends from the trocar hub 131. The trocar hub 131 and the trocar shaft 132 can collectively define a channel for receiving surgical instruments and/or imaging device (e.g., imaging device 192). In some embodiments, the trocar 130 can be coupled to an obturator to enable initial placement of the trocar 130 into the body lumen and/or the cavity. In some embodiments, the trocar 130 can include one or more gas channels or vents that can allow for delivery of gases (e.g., insufflation) and/or escape of gases.

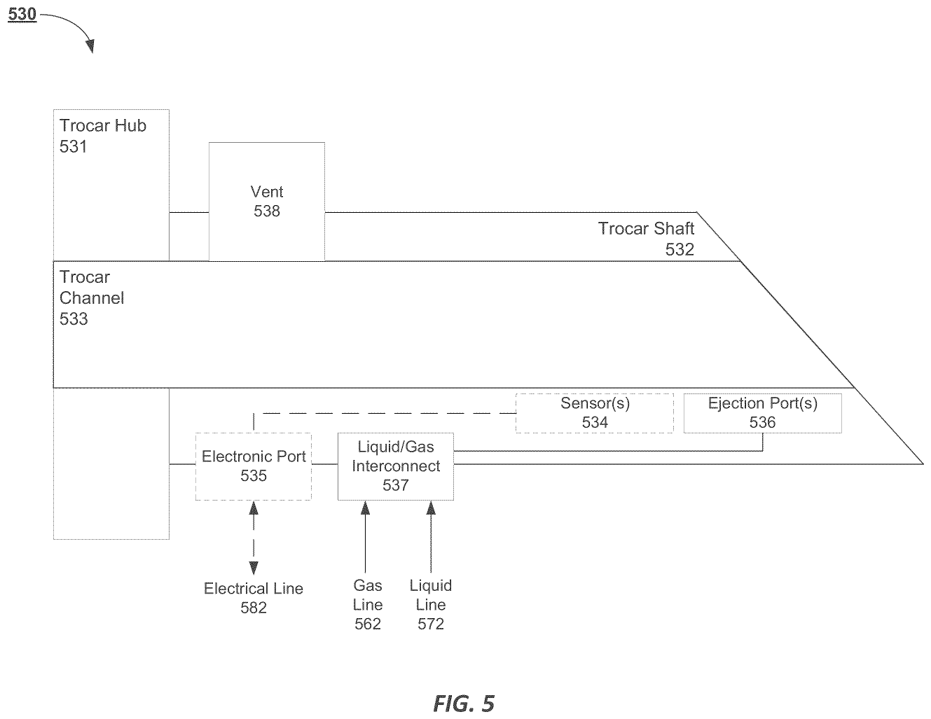

Additionally, the trocar 130 can include one or more channels or lines in fluidic communication with the fluid delivery system 110, e.g., for receiving gas and/or liquid from the fluid delivery system 110. The fluid delivery system 110 can be configured to control delivery of gas and/or liquid for performing one or more wash sequences, e.g., to clean a distal end of the imaging device. In some embodiments, the channels or lines can be primed after each deployment of wash solution (e.g., after each wash sequence). In some embodiments, the trocar 130 can be maintained in place by an anchor (not shown) placed on the outside of the patient's body. In some embodiments, the trocar 130 can optionally include one or more sensors to detect a presence, a position, and/or orientation of a surgical instrument and/or imaging device (e.g., imaging device 192). Further details of an example trocar are provided with reference to FIGS. 5-7 below.

The imaging system 190 can be coupled to the trocar 130. In some embodiments, at least a portion of the imaging system 190 can be disposed and/or positioned within the trocar 130. For example, the imaging system 190 can include the imaging device 192 (e.g., scopes such as endoscope, laparoscope, fiberscope, etc.). The trocar 130 can be configured to receive at least a portion of the imaging device 192 (e.g., via the trocar channel). Alternatively, the imaging device 192 can be separate from the imaging system 190. For example, the imaging system 192 can be positioned entirely outside the trocar 130 while at least a portion of the imaging device 192 can be positioned within the trocar 130. In such embodiments, the imaging device 192 can be coupled (e.g., communicably coupled and/or operably coupled) to the imaging system 190.

The imaging device 192 can be any suitable imaging device configured to be inserted into an interior portion of a subject's anatomy (e.g., body lumen or cavity) to acquire image data of the body lumen, cavity, and/or internal organs and transmit the image data to a user (e.g., surgeon, operator, etc.) for examination. For example, the imaging device 192 can be any suitable scope such as endoscope, laparoscope, fiberscope, a combination thereof, and/or the like. Typically, the imaging device 192 can include an elongate body that can be inserted into a body lumen and/or cavity of a subject with an image capturing device at one end of the elongate body. The image capturing device can be configured to acquire images of the body lumen, cavity, and/or the internal organs. In some embodiments, the imaging device 192 can be similar to the endoscope described in U.S. Patent Publication No. 2021/0127963, incorporated above by reference.

The image data from the imaging device 192 can be transmitted to the user via a user interface of the imaging system 190. For example, in some embodiments, the imaging system 190 can include a display. The image data from the imaging device 192 can be displayed on the display for the user to examine. The imaging system 190 can include a processor to process and analyze image data from the imaging device 192. The image data and/or the output of the analysis can be stored in a memory of the imaging system 190. In some embodiments, as discussed above, the imaging system 190 can include a user interface to display the image data acquired from the imaging device 192 to a user.

The robotic system 180 can be communicably coupled to the imaging system 190. For example, the robotic system 180 can be communicably coupled to the imaging system 190 via any suitable type of network (e.g., e.g., a local area network (LAN), a wide area network (WAN), a virtual network, a telecommunications network) implemented as a wired network and/or wireless network. In some embodiments, at least a portion of the imaging system 190 can be operably coupled to the robotic system 180. For example, the imaging device 192 can be operably coupled to the robotic system 180 such that the robotic system 180 can be configured to position and orient the imaging device 192 during surgical procedure.

In some embodiments, the robotic system 180 can include a compute device configured to receive the image data and/or analysis of the image data from the imaging system 190, process the image data and/or analysis from the imaging system 190, and control and/or transmit instructions to control at least the fluid delivery system 110 and/or the imaging device 192. In some embodiments, the compute device can be configured to obtain and analyze sensor data from sensors within the trocar 130 and/or from sensors external to the trocar 130. For example, the compute device can be configured to analyze the data from the imaging system 190 and/or sensor data from the sensors. Based on the analysis, the compute device can be configured to determine: the position and/or orientation of the imaging device 192 during the surgical procedure, position and/or orientation of the imaging device 192 relative to the trocar 130 during cleaning and during the surgical procedure, whether the imaging device 192 requires to be cleaned during the surgical procedure, the time period and/or time points at which the imaging device 192 requires to be cleaned during the surgical procedure, the position and/or orientation of the imaging device for cleaning the imaging device 192, the type of cleaning and/or the intensity of cleaning required by the imaging device 192, and/or the like.

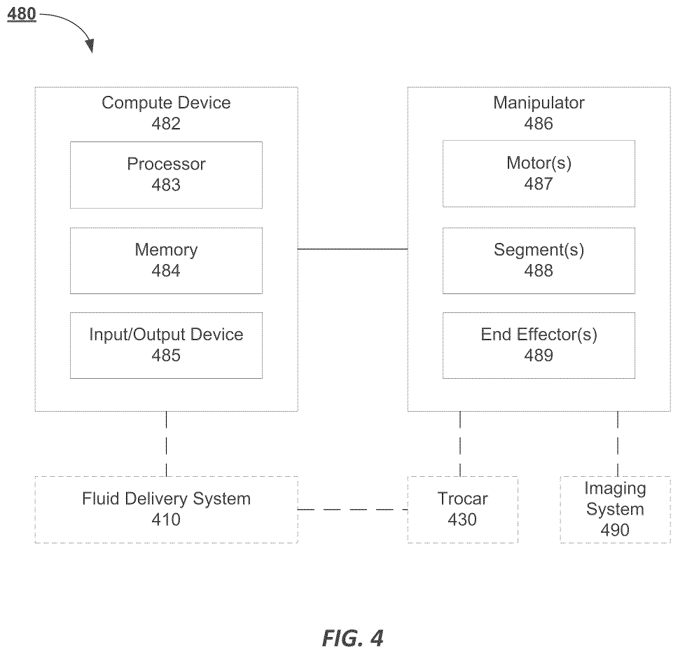

In some embodiments, the robotic system 180 can include a manipulator configured to manipulate a surgical device (e.g., trocar 130), the imaging device 192, the imaging system 190, and/or the fluid delivery system 110. For example, in some embodiments, the robotic system 180 can be configured to perform at least a portion of the surgical procedure. Accordingly, the manipulator can be configured to insert the trocar 130 into the body lumen/cavity and/or initially position the imaging device 192 in the trocar 130. The manipulator can be configured to insert the trocar 130 and/or initially position the imaging device 192 relative to the trocar 130 automatically (e.g., based on instructions from the compute device of the robotic system 180). Additionally or alternatively, the manipulator can be configured to insert the trocar and/or initially position the imaging device 192 based on instructions from a user. In some embodiments, the manipulator can be configured to position the imaging device 192 relative to the trocar 130 (e.g., automatically based on instructions from the compute device of the robotic system 180) for cleaning the imaging device 192. In some embodiments, the manipulator can be configured to manipulate the fluid delivery system 110 (e.g., an actuator in the fluid delivery system 110, a pump in the fluid delivery system 110, etc.) or send signals or instructions to a fluid delivery system 110 to clean the imaging device 192. Further details of an example robotic system 180 is provided with reference to FIG. 4 below.

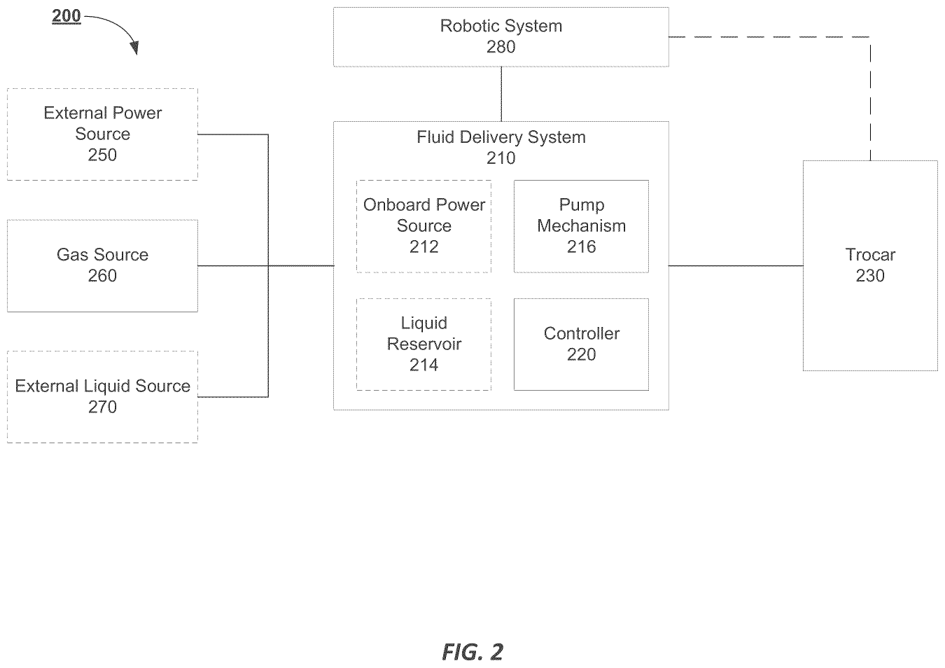

The robotic system 180 can be coupled (e.g., communicably coupled and/or operably coupled) to the fluid delivery system 110. The fluid delivery system 110 can be fluidly coupled with the trocar 130. The fluid delivery system 110 can include or be coupled to one or more of a pump mechanism, a controller, a power source, or a liquid reservoir. The power source can be configured to deliver power to the pump mechanism and the controller. The liquid reservoir can be configured to contain a liquid for cleaning the imaging device 192. The pump mechanism can be configured to aid in the delivery of the liquid to the trocar 130 for cleaning the imaging device 192. The controller can be configured to control the operation of the pump mechanism. Further details of an example fluid delivery system 110 is provided with reference to FIG. 2 below.

The fluid delivery system 110 can be fluidly coupled to the gas source 160. The gas source 160 can be used to pressurize the liquid and deliver the liquid via the fluid delivery system 110 to clean the imaging device 192. In some embodiments, the gas source 160 can include a container (e.g., a tank) that houses a volume of pressurized gas. In some embodiments, the gas source 160 can deliver gas at a pressure of between about 20 psi and about 50 psi, including all values and sub-ranges therebetween. In some embodiments, the gas delivered by the gas source 160 can include CO₂, nitrogen, argon, or any other inert gas or combinations thereof. The selected gas can be a gas that is commonly used in medical procedures and is safe for delivery into a body lumen and/or cavity.

As disclosed above, in some embodiments, the fluid delivery system 110 can include a liquid reservoir with liquid configured to wash the imaging device 192. Additionally or alternatively, the fluid delivery system 110 can be coupled to an external liquid source 170. The external liquid source 170 can be used to supply liquid to the fluid delivery system 110 and/or directly into one or more liquid lines. The external liquid source 170 be separate from the fluid delivery system 110 but coupled to the fluid delivery system 110, e.g., via a fluid line and/or a port. In some embodiments, the external liquid source 170 can be a fluid bag or other type of fluid containing element. In some embodiments, the external liquid source 170 can be a water line or other fluid line within a building that can be coupled via a faucet or other connection to the fluid delivery system 110. In some embodiments, the external liquid source 170 can be used to fill (e.g., pre-fill or re-fill) the liquid reservoir included in the fluid delivery system 110.

FIG. 2 depicts a system 200 for delivering fluid to a trocar 230 (e.g., structurally and/or functionally similar to trocar 130 in FIG. 1), according to an embodiment. As shown, the system 200 includes a fluid delivery system 210 (e.g., structurally and/or functionally similar to fluid delivery system 110 in FIG. 1) fluidically coupled to a trocar 230 and a gas source 260 (e.g., structurally and/or functionally similar to gas source 160 in FIG. 1). The fluid delivery system 210 can optionally include an onboard power source 212. Alternatively or additionally, the fluid delivery system 210 can optionally be coupled to an external power source 250. The fluid delivery system 210 includes a pump mechanism 216 and a controller 220. The fluid delivery system 210 can optionally include a liquid reservoir 214. Alternatively or additionally, the fluid delivery system 210 can optionally be coupled to an external liquid source 270 (e.g., structurally and/or functionally similar to external liquid source 170 in FIG. 1). The fluid delivery system 210 can be coupled (e.g., communicably coupled and/or operably coupled) to a robotic system 280 (e.g., structurally and/or functionally similar to robotic system 180 in FIG. 1). Lines depicted in FIG. 2 that connect various units of the system 200 can represent electrical, physical, and/or fluidic couplings.

The onboard power source 212 can be an optional component integrated into the fluid delivery system 210. The onboard power source 212 can be configured to power the pump mechanism 216 and/or the controller 220. In some embodiments, the onboard power source can include a battery. In some embodiments, the onboard power source 212 can include a fuel cell. In some embodiments, the onboard power source can be integrated into the same structure as the liquid reservoir 214, the pump mechanism 216, and/or the controller 220. For example, the onboard power source 212, the liquid reservoir 214, and the pump mechanism 216 can be disposed together in a housing (or one or more housing sections that couple together to form a housing).

Optionally, an external power source 250 can be coupled to the fluid delivery system 210 to deliver power to one or more components of the fluid delivery system 210. In some embodiments, the external power source 250 can include a wall outlet. In some embodiments, the external power source 250 can include a battery or a battery pack physically separated from the fluid delivery system 210. In some embodiments, the external power source 250 can power the pump mechanism 216, the controller 216, and/or the onboard power source 212.

The liquid reservoir 214 can be an optional component integrated into the fluid delivery system 210. The liquid reservoir 214 can be configured to contain a liquid (e.g., wash liquid or solution), e.g., for cleaning an imaging device (e.g., imaging device 192 in FIG. 1). In some embodiments, the washing fluid can include a saline solution, a buffered solution, a bio-compatible surfactant, and/or any other suitable wash solution, including those described in U.S. Patent Publication No. 2021/0127963. The liquid reservoir 214 can be configured to contain a volume of liquid that is sufficient for conducting at least about 5, at least about 10, at least about 50, at least about 100, at least about 500, at least about 1000, at least about 1500, or at least about 2000 wash sequences. For example, the liquid reservoir 214 can be filled with enough liquid for cleaning an imaging device (e.g., imaging device 192 in FIG. 1) throughout the duration of a surgical procedure. In some embodiments, the liquid reservoir 214 can be pre-filled with different volumes of liquid, e.g., depending on the estimated number of times that an imaging device positioned within a body lumen and/or cavity may need to be cleaned. Therefore, for longer procedures that may require a greater number of wash sequences, the liquid reservoir 214 may be filled with a greater volume of liquid. In some embodiments, the liquid reservoir 214 can have a volume of at least about 5 mL, at least about 10 mL, at least about 15 mL, at least about 20 mL, at least about 25 mL, at least about 30 mL, at least about 35 mL, at least about 40 mL, at least about 45 mL, at least about 50 mL, at least about 55 mL, at least about 60 mL, at least about 65 mL, at least about 70 mL, at least about 75 mL, at least about 80 mL, at least about 85 mL, at least about 90 mL, or at least about 95 mL. In some embodiments, the liquid reservoir 214 can have a volume of no more than about 100 mL, no more than about 95 mL, no more than about 90 mL, no more than about 85 mL, no more than about 80 mL, no more than about 75 mL, no more than about 70 mL, no more than about 65 mL, no more than about 60 mL, no more than about 55 mL, no more than about 50 mL, no more than about 45 mL, no more than about 40 mL, no more than about 35 mL, no more than about 30 mL, no more than about 25 mL, no more than about 20 mL, no more than about 15 mL, or no more than about 10 mL. Combinations of the above-referenced volumes of the liquid reservoir 214 are also possible (e.g., at least about 5 mL and no more than about 100 mL or at least about 20 mL and no more than about 40 mL), inclusive of all values and ranges therebetween. In some embodiments, the liquid reservoir 214 can have a volume of about 5 mL, about 10 mL, about 15 mL, about 20 mL, about 25 mL, about 30 mL, about 35 mL, about 40 mL, about 45 mL, about 50 mL, about 55 mL, about 60 mL, about 65 mL, about 70 mL, about 75 mL, about 80 mL, about 85 mL, about 90 mL, about 95 mL, or about 100 mL. Alternatively or additionally, an external liquid source 270 (as described above) can be used to supply liquid to the fluid delivery system 210.

The pump mechanism 216 can aid in delivering liquid (e.g., wash liquid or solution) to the trocar 230. In some embodiments, the pump mechanism 216 can include or form part of a centrifugal pump, peristaltic pump, lobe pump, rotary gear pump, horizontal split case pump, air operated pump, diaphragm pump, magnetically driven pump, a mechanically driven pump, an electrically driven pump, or any other suitable pump apparatus or combinations thereof. In a specific embodiment, the pump mechanism 216 can include a plunger, platform, shaft, or other suitable component that can be actuated (e.g., via a pump actuator 326) to compress a fluid line to deliver a liquid. For example, a pump mechanism 216 implemented as a plunger can be actuated to compress a flexible housing or tubing that contains a liquid. The compression of the flexible housing or tubing can cause the liquid within the flexible housing or tubing to be driven toward the trocar 230, e.g., to fill the lines for a wash or cleaning sequence.

The controller 220 can control the operation of the pump mechanism 216. In some embodiments, the controller 220 can be in communication with or include a processor and/or a user interface. Operation of the pump mechanism 216 can be automatic or user-controlled. In some embodiments, the user via the user interface can set parameters for when to activate the pump mechanism 216, e.g., to supply additional liquid for cleaning an imaging device. In some embodiments, the controller 220 can activate the pump mechanism 216 after each wash sequence to fill the liquid lines for a subsequent wash sequence. In some embodiments, the controller 220 can activate the pump mechanism 216 to fill the liquid lines in response to an indication that an imaging device has been positioned for cleaning (e.g., based on signals received by the controller 220 from one or more sensors). In some embodiments, the controller 220 can activate the pump mechanism 216 to fill the liquid lines in response to a detection of a drop in pressure or volume in the liquid lines (e.g., based on signals received by the controller 220 from one or more sensors). As discussed above, the gas source 260 can be used to pressurize the liquid and deliver the liquid via the fluid delivery system 210.

The robotic system 280 can be operatively coupled to the fluid delivery system 210. The robotic system 280 can also be operatively coupled to an imaging device (e.g., imaging device 192). The robotic system 280 can be configured to analyze image data from a system including the imaging device to determine whether the imaging device needs cleaning. In some embodiments, the robotic system 280 can be configured to indicate when to initiate a wash sequence. For example, the robotic system 280 can display via a user interface an indication to initiate the wash sequence. In such embodiments, a user can then instruct the fluid delivery system 210 to initiate the wash sequence. Additionally or alternatively, the robotic system 280 can be configured to automatically initiate a wash sequence. For example, the robotic system 280 can be configured to transmit instructions to the controller 220 of the fluid delivery system 210 to initiate the wash sequence. The controller 220 can be configured to initiate the wash sequence based on the instructions received from the robotic system 280 (e.g., by controlling the pump mechanism 216 to prime a liquid line with liquid, by activating gas delivery to propel the delivery of a predetermined volume of liquid, etc.). In some embodiments, a manipulator included in the robotic system 280 can be configured to automatically initiate the wash sequence when the imaging device needs cleaning. For example, an end effector of the manipulator can be configured to provide an input into the fluid delivery system 210 (e.g., depress a button, select an icon in a touchscreen interface, or mechanically provide some other input) such that the fluid delivery system 210 can initiate the wash sequence when the robotic system 280 determines that the imaging device needs cleaning.

In some embodiments, the robotic system 280 can be configured to indicate the type of cleaning and/or the intensity of cleaning (e.g., via a user interface). In such embodiments, a user can control the fluid delivery system 210 so as to control an amount of fluid delivered for cleaning, a pressure at which the fluid is delivered for cleaning, and/or the time duration during which the fluid is delivered for cleaning. Additionally or alternatively, the robotic system 280 can be configured to transmit instructions to the controller 220 to control the fluid delivery system 210 so as to control an amount of fluid delivered for cleaning, a pressure at which the fluid is delivered for cleaning, and/or the time duration during which the fluid is delivered for cleaning. In some embodiments, the manipulator included in the robotic system 280 can be configured to automatically control the fluid delivery system 210 so as to control an amount of fluid delivered for cleaning, a pressure at which the fluid is delivered for cleaning, and/or the time duration during which the fluid is delivered for cleaning.

In some embodiments, the robotic system 280 can be optionally coupled to the trocar 230, e.g., for receiving data and/or sending data to the trocar 230. For example, the robotic system 280 can be configured to receive data from one or more sensors located in the trocar 230. Alternatively or additionally, a manipulator of the robotic system 280 can be coupled to the trocar 230, e.g., for manipulating the trocar 230 such as during initial insertion of the trocar 230, removal of the trocar 230, etc. and/or for maintaining a position of the trocar 230.

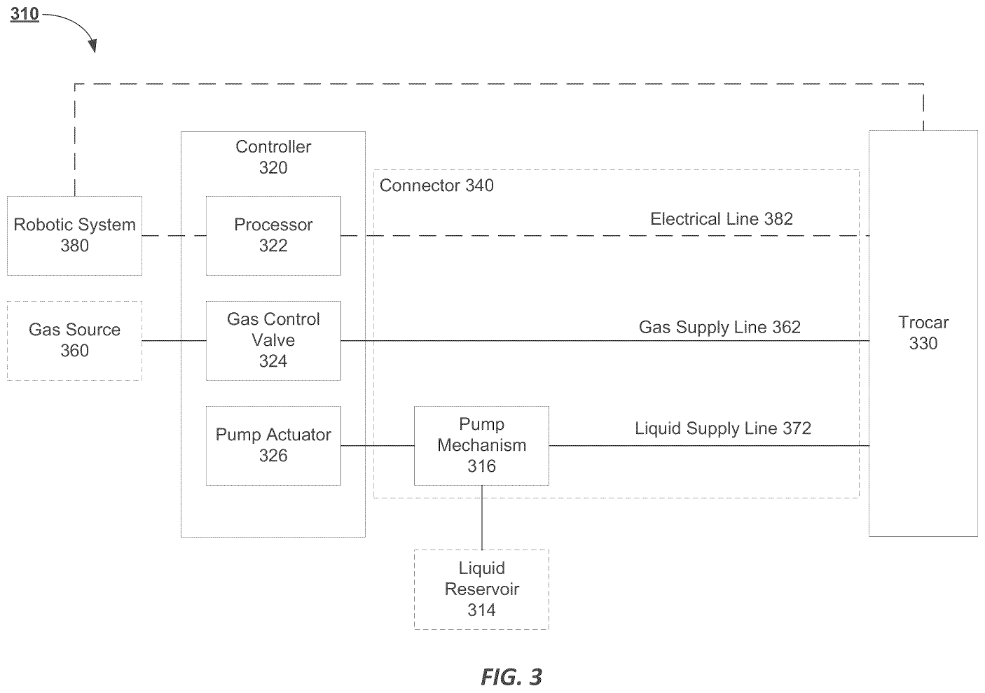

FIG. 3 provides a more detailed view of fluid connections between fluid sources (gas source 360, liquid reservoir 314), controller 320, connector 340, and trocar 330 of a cleaning system or fluid delivery system 310, according to an embodiment. The fluid delivery system 310 can be structurally and/or functionally similar to other fluid delivery systems described herein, including, for example, fluid delivery system 110 in FIG. 1 and fluid delivery system 210 in FIG. 2. As shown, the fluid delivery system 310 includes a controller 320. The fluid delivery system 310 can be fluidically coupled to the trocar 330 (e.g., structurally and/or functionally similar to trocar 130 in FIG. 1) and an optional gas source 360 (e.g., structurally and/or functionally similar to gas source 160 in FIG. 1). The controller 320 can include a processor 322, a gas control valve 324, and a pump actuator 326. A liquid reservoir 314 can be fluidically coupled to a liquid supply line 372, with a pump mechanism 316 disposed along the coupling or line to control delivery of the liquid. A gas source 360 can be fluidically coupled to a gas supply line 362, with a gas control valve 324 disposed along the coupling or line to control delivery of the gas. In some embodiments, a robotic system 380 (e.g., structurally and/or functionally similar to robotic system 180 in FIG. 1 and robotic system 280 in FIG. 2) can be electronically and/or communicably coupled to the processor 322 to control the wash sequence for an imaging device.

In some embodiments, an optional connector 340 can house the gas supply line 362, the liquid supply line 372, and the electrical line 382. In some embodiments, the connector 340 can include an outer cylindrical or tubular housing that defines a lumen for containing the gas supply line 362, the liquid supply line 372, and the electrical line 382. In some embodiments, the connector 340 can be composed of an insulative material, a rubber, a plastic, a polymer, or any combination thereof. The connector 340 can include a proximal connection or controller connection and a distal connection or trocar connection that each include connecting elements for coupling to the controller 320 and the trocar 330, respectively. In some embodiments, the connector 340 can be a cable that houses the gas supply line 362, the liquid supply line 372, and the electrical line 382.

The processor 322 can be coupled to an electrical line 382. In some embodiments, the trocar 330 can include one or more electrical elements (e.g., sensors) disposed in the trocar 330. In such embodiments, the processor 322 can be coupled to the electrical line 382 to send and/or receive data from the electrical elements disposed in the trocar 330. In some embodiments, one or more electrical elements (e.g., sensors) can be disposed external to the trocar 330. In such embodiments, the processor 322 can be coupled to the electrical line 382 to send and/or receive data from these electrical elements disposed external to the trocar 330. Electrical elements such as sensors (disposed internal and/or external to the trocar 330) can be configured to detect when an imaging device (e.g., imaging device 192) is being retracted within the trocar for initiating a wash sequence. For example, one or more sensors disposed internal and/or external to the trocar 330 can detect a position of the distal end of an imaging device positioned within the trocar 330. When the sensor(s) detect that the imaging device is retracted and/or positioned for cleaning, the sensor(s) can send that data to the processor 322, which can activate the delivery of gas and/or liquid to clean the distal end of the imaging device.

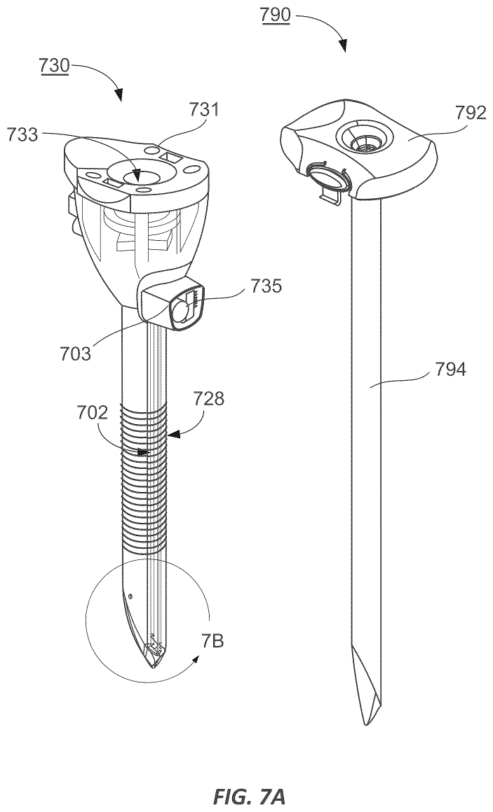

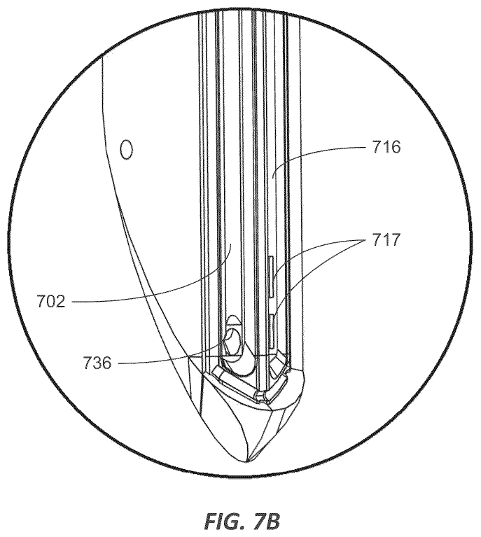

Optionally, in some embodiments, the processor 322 can be coupled to the robotic system 380 to send information to and/or receive information from the robotic system 380. For example, the robotic system 380 can analyze image data from the imaging device and detect when to initiate a wash sequence for the imaging device. The processor 322 can receive data relating to the wash sequence from the robotic system 380 and activate the delivery of gas and/or liquid to clean the distal end of the imaging device. Alternatively, in some embodiments, the processor 322 of the cleaning system 310 may not be operatively coupled to a robotic system. In such embodiments, the processor 322 can be configured to control the operation of the cleaning system 310 (e.g., delivery of liquid and/or gas) based on signals received from one or more sensors coupled to the trocar 330, as further described with reference to FIGS. 5 and 7A-7B.

In some embodiments, the processor 322 can be operatively coupled to one or more electrical components of the trocar 330 (e.g., one or more sensors) via an electrical line 382. The electrical line 382 can transmit sensor signals and/or data to the processor 322, e.g., to cause the processor 322 to initiate delivery of liquid and/or gas for cleaning an endoscope. In some embodiments, the electrical line 382 can include conductive wiring. In some embodiments, the conductive wiring can be composed of copper, silver, brass, gold, titanium, stainless steel, carbon steel, or any combination thereof. In some embodiments, the electrical line 382 can be housed within the connector 340 and insulated from external elements.

The gas source 360 and the gas control valve 324 can be fluidically coupled to the trocar 330 via the gas supply line 362. The gas control valve 324 can control delivery of gas into the trocar 330. The gas control valve 324 can be controlled by the controller 320 (specifically, processor 322) to deliver gas at desired times, e.g., when an imaging device is positioned for cleaning. In some embodiments, the gas supply line 362 can include flexible tubing. In some embodiments, the tubing can be composed of a polymer, polyvinylchloride (PVC), polyurethane, Tygon®, acrylic, or any other suitable material.

The pump actuator 326 can actuate the pump mechanism 316 to deliver liquid to the trocar 330 via the liquid supply line 372. The pump actuator 236 can include an electrical motor and/or other drive mechanisms for actuating the pump mechanism 316. In some embodiments, the pump mechanism 316 can be a plunger, shaft, or other suitable structure that can be actuated to compress flexible tubing containing the liquid, e.g., to pump the liquid. In some embodiments, the pump actuator 326 can be powered by a battery, a wall outlet, or any other suitable power mechanism. In some embodiments, the liquid supply line 372 can include flexible tubing. In some embodiments, the tubing can be composed of a polymer, PVC, polyurethane, Tygon®, acrylic, or any other suitable material.

The robotic system 380 can be coupled to an imaging system including an imaging device that is insertable into the trocar 330. The robotic system 380 can be configured to detect when the imaging device needs to be cleaned and in response to such detection, initiate a wash sequence. Optionally, the robotic system 380 can also be coupled to the trocar 330, e.g., for sending or receiving data to or from the trocar 330, for manipulating the trocar 330, and/or for maintaining a position of the trocar 330.

FIG. 4 provides detailed view of a robotic system 480 (e.g., structurally and/or functionally similar to robotic systems 180, 280, and 380 or any other robotic systems described herein) for cleaning an imaging device (e.g., imaging device 192 in FIG. 1). The robotic system 480 can include a compute device 482 that can be coupled (e.g., communicably coupled and/or operably coupled) to a manipulator 486. The compute device 482 can include a processor 483, a memory 484, and input/output devices 485. The manipulator 486 can include one or more motors 487, one of more segments 488, and one or more end effectors 489.

The compute device 482 can be configured to control the manipulator 486 to manipulate a surgical instrument, an imaging device, and/or the fluid delivery system. The compute device 482 can be any suitable processing device configured to run and/or execute certain functions. Some non-limiting examples of the compute device 482 include computers (e.g., desktops, workstations, personal computers, laptops etc.), tablets (e.g., Apple iPad®, Samsung Galaxy® Tab, Microsoft Surface®, etc.), mobile devices and smart phones (e.g., Apple iPhone®, Samsung Galaxy®, Google Pixel®, etc.), etc.

The compute device 482 can include a processor 483 that can be configured to determine: the position and/or orientation of the imaging device during the surgical procedure, position and/or orientation of the imaging device relative to the trocar 430 during cleaning and during the surgical procedure, whether the imaging device requires to be cleaned during the surgical procedure, the time period and/or time points at which the imaging device requires to be cleaned during the surgical procedure, the position and/or orientation of the imaging device in order to clean the imaging device, the type of cleaning and/or the intensity of cleaning required by the imaging device, and/or the like. In some embodiments, the processor 483 can be configured to control the manipulator 486 based on one or more of these determinations. For example, based on determining that the imaging device needs to be cleaned, the processor 483 can be configured to control the manipulator 486 to position the imaging device at a specific position and/or orientation relative to the trocar 430 in order to initiate a wash sequence. In a similar manner, the processor 483 can be configured to control the manipulator 486 to position the imaging device at a specific position/or orientation relative to the trocar 430 after the wash sequence to continue the surgical procedure. In these examples, the processor 483 can be configured to control the manipulator 486 to vary the position and/or orientation of the trocar 430 and/or the imaging device. In some embodiments, the processor 483 can be configured to control the fluid delivery system 410 based on its determinations. For example, the processor can be configured to control the fluid delivery system 410 so as to initiate or control a wash sequence (e.g., select a type of wash and/or intensity of the wash) for the imaging device.

The processor 483 can be any suitable processing device(s) configured to run and/or execute a set of instructions or code. For example, the processor 483 can be and/or can include one or more data processors, image processors, graphics processing units (GPU), physics processing units, digital signal processors (DSP), analog signal processors, mixed-signal processors, machine learning processors, deep learning processors, finite state machines (FSM), compression processors (e.g., data compression to reduce data rate and/or memory requirements), encryption processors (e.g., for secure wireless data and/or power transfer), and/or central processing units (CPU). The processor can be, for example, a general-purpose processor, microprocessor, microcontroller, Field Programmable Gate Array (FPGA), an Application Specific Integrated Circuit (ASIC), a processor board, and/or the like. The processor 483 can be configured to run and/or execute application processes and/or other modules, processes and/or functions associated with the system 100. The underlying device technologies may be provided in a variety of component types (e.g., metal-oxide semiconductor field-effect transistor (MOSFET) technologies like complementary metal-oxide semiconductor (CMOS), bipolar technologies like generative adversarial network (GAN), polymer technologies (e.g., silicon-conjugated polymer and metal-conjugated polymer-metal structures), mixed analog and digital, and/or the like.

The memory 484 can be any suitable memory device(s) configured to store data, information, computer code or instructions (such as those described above), and/or the like. In some embodiments, the memory 484 can be and/or can include one or more of a random access memory (RAM), static RAM (SRAM), dynamic RAM (DRAM), a memory buffer, an erasable programmable read-only memory (EPROM), an electrically erasable read-only memory (EEPROM), a read-only memory (ROM), flash memory, volatile memory, non-volatile memory, combinations thereof, and the like. In some embodiments, the memory 484 can store instructions to cause the processor to execute modules, processes, and/or functions associated with the system 100, such as determining when to initiate a wash sequence, positioning of the imaging device, controlling of the fluid delivery system, etc.

An I/O device 485 can include any suitable input device that can be configured to receive inputs from the user and/or any suitable output device that can be configured to send outputs to the manipulator 486, the trocar 430, the imaging system 490, the fluid delivery system 410, and/or the user. In some embodiments, the I/O device 485 can be a user control such as a joystick, a remote user control, keyboard, trackball, etc. that can receive input from the user. In some embodiments, the I/O device 485 can be an audio device such as a microphone and/or a speaker that receives audio input from the user. In such embodiments, the I/O device 485 can be a display device (e.g., a display, a touch screen, a microphone, etc.) that displays output to the user and/or receives inputs from a user (e.g., via a touchscreen interface).

In some embodiments, the compute device 482 can transmit signals (e.g., output) to the manipulator 486 to control the manipulator 486 in order to control the fluid delivery system 410, the trocar 430, and/or the imaging device of the imaging system 490. The manipulator 486 can include two or more segments 488 that can be coupled together via joints. Joints can allow one or more degrees of freedom. For example, joints can provide for translation along and/or rotation about one or more axes. The manipulator 486 can include one or more end effectors 489 to engage and/or interact with surgical instruments (e.g., trocar 430), imaging system 490, fluid delivery system 410, etc. For example, manipulator 486 can include a gripping mechanism that can releasably engage (e.g., grip) the trocar 430 and/or the imaging device such that the manipulator can hold the trocar 430 and/or position or orient the imaging device relative to the trocar 430 prior to, during, or after a wash sequence. Other examples of end effectors include, for example, vacuum engaging mechanism(s), magnetic engaging mechanism(s), suction mechanism(s), and/or combinations thereof. In some embodiments, the manipulator 486 can include more than one end effector 489 such that each end effector is configured to interact and/or engage with one of the trocar 430, the fluid delivery system 410, and/or the imaging device respectively. For example, the manipulator 486 can include two end effectors 489. A first end effector can be configured to interact and/or engage with the imaging device so as to control the movement of the imaging device during washing. Additionally or alternatively, a second end effector can be configured to interact and/or engage with the trocar 430 so as to maintain the positioning of the trocar 430.

The manipulator 486 (e.g., the segments 488 and/or the end effector(s) 489) can be actuated by one or more motor(s) 487. Put differently, the motor 487 may actuate the segments 488 and/or the end effector(s) 489 of the manipulator 486 so that the manipulator 486 moves. In some embodiments, the manipulator 486 can include one or more sensors to measure sensory information, including information relating to the manipulator 486. Examples of sensors include image capture devices, position encoders, torque and/or force sensors, touch and/or tactile sensors, etc. The sensors can be disposed on or integrated with either the segments 488, or the joints, or a combination of both. The sensory information can be transmitted to one or more I/O device(s) 485 so that subsequent movement of the manipulator 486 can be based on the sensory information.

In some embodiments, the robotic system 480 can be or form part of a robotic system for performing a surgical procedure or operating an imaging device or imaging system 490. For example, the robotic system 480 can be integrated into a robotic system that is configured to perform a surgical procedure. The robotic system 480 can be configured to insert a trocar into an interior portion of the subject's anatomy (e.g., body lumen and/or cavity) and to maintain the position of the trocar within the interior portion. The robotic system can then insert an imaging device into the body lumen and/or cavity via a channel of the trocar. The robotic system 480 can position the imaging device for capturing a view of the body cavity, e.g., for providing visual information to a surgeon and/or robotic system. The robotic system 480 can monitoring the image or video feed from the imaging device, and in response to determining that the imaging device needs to be cleaned, retract the imaging device for cleaning (e.g., via the trocars of the cleaning systems as described herein). As such, systems, devices, and methods described herein contemplate the adaptation of surgical robotic systems to include the functionality of cleaning, e.g., using a trocar-based cleaning system. Such surgical robotic systems can be calibrated on how to position and/or orient an imaging device relative to a trocar of a cleaning system, how to determine when a wash sequence is needed, what type of wash sequence to select, and/or the like.

FIG. 5 provides detailed view of a trocar 530 (e.g., structurally and/or functionally similar to trocar 130 in FIG. 1), according to an embodiment. As shown, the trocar 530 can include a trocar hub 531 and a trocar shaft 532. A trocar channel 533 can extend through the trocar shaft 532. One or more ejection ports 336, and/or a liquid/gas interconnect 337 can be integrated into or disposed in the trocar shaft 332. In some embodiments, one or more sensor 534 and/or an electronic port 535 can be optionally included in the trocar shaft 532. A vent 538 can also be integrated into or disposed in the trocar shaft 332. An electrical line 582 can be coupled to the electronic port 535, while a gas line 562 and a liquid line 572 can be coupled to the liquid/gas interconnect 537. In some embodiments, the gas line 562, the liquid line 572, and the electrical line 582 can be the same or substantially similar to the gas supply line 362, the liquid supply line 372, and the electrical line 382, as described above with reference to FIG. 3. Thus, certain aspects of the gas line 562, the liquid line 572, and the electrical line 582 are not described in greater detail herein.

The trocar hub 531 is an enlarged portion of the trocar 530 for housing one or more components of the trocar 530. The trocar hub 531 can provide a handle for placement of the trocar 530. The trocar shaft 532 is an elongated portion of the trocar 530 and can be connected to the trocar hub 531. During use, the trocar hub 531 can be positioned outside of a subject's body while the trocar shaft 532 (or a substantial majority of the trocar shaft 532) can be positioned within the subject's body.

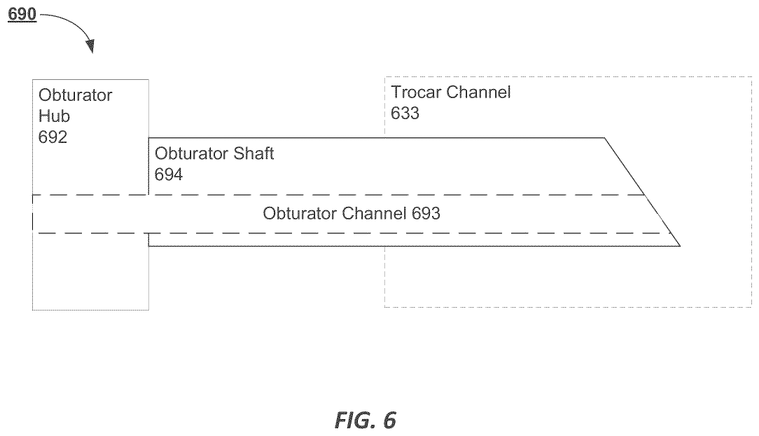

The trocar hub 531 and the trocar shaft 532 can collectively define a trocar channel 533 for receiving an instrument, e.g., an imaging device, an obturator, etc. In some embodiments, the trocar channel 533 can have a diameter of between about 1 mm (3 French) and about 10 mm (30 French), including all sub-ranges and values therebetween. For example, the trocar channel 533 can have a diameter of about 10 French or slightly larger than 10 French such that the trocar channel 533 can be configured to receive an instrument (e.g., endoscope) having up to a 10 French diameter. During use, the trocar 530 can be positioned within a patient such that the channel 533 extends into a body lumen and/or a cavity. The channel can therefore provide access to a body lumen and/or a cavity, e.g., for positioning one or more instruments within the body lumen and/or the cavity. The trocar 530 can be positioned through an incision in the subject's body. In some embodiments, an obturator (e.g., obturator 690 in FIG. 6) can be positioned within the trocar channel 533 while the trocar is being positioned within the body and then removed after the distal end of the trocar has been positioned within the body lumen and/or the cavity. Other instruments (e.g., imaging devices) can then be positioned within the trocar channel 533 after the obturator has been removed. Further details of an obturator are described with reference to FIG. 6.

The trocar 530 can form a part of a cleaning system for an imaging device, e.g., such as the system described above with reference to FIG. 1. As such, the trocar 530 can include components that can facilitate a cleaning or wash sequence associated with an imaging device. In particular, the trocar 530 can include one or more ports (e.g., electronic port 535, ejection port(s) 536, liquid/gas port(s)) and/or one or more sensor(s) 534.

In some embodiments, the trocar 530 can optionally include a liquid/gas interconnect 537. The liquid/gas interconnect 537 can be configured to combine a liquid stream and a gas stream into one output stream. The liquid/gas interconnect 537 receives a feed from the gas line 562 and the liquid line 572. The liquid/gas interconnect 537 can include a collection of valves and tubes for controlling the delivery of fluid (e.g., gas and/or liquid). In some embodiments, the liquid/gas interconnect 537 can be integrated into or disposed in a connector that coupled to the trocar 530 (e.g., a trocar connection or distal connection of connector 340) instead of being integrated into or disposed in the trocar 530. In such embodiments, the output stream from the liquid/gas interconnect 537 can be coupled to a liquid/gas port integrated into or disposed in the trocar shaft 532.

In some embodiments, the trocar 530 can optionally include one or more sensors 534. The sensor(s) 534 can detect whether a device (e.g., an obturator, an imaging device) is in the trocar channel 533 and/or a position and/or orientation of the device within the trocar channel 533. The sensor(s) 534 can trigger liquid and/or gas deployment via the ejection port(s) 536 upon detecting that the device is in a position and/or orientation for cleaning. For example, when a device is retracted into the trocar channel 533 such that at least one sensor 534 detects the device, the sensor(s) 534 can trigger liquid and/or gas deployment. The sensor(s) 534 can be coupled to a controller (e.g., controller 220) via electronic port 535 and electrical line 582. As such, the sensor(s) 534 can send signals to the controller for detecting a position and/or orientation of the device. In response to detecting that the device is in a position and/or orientation for cleaning, the controller can trigger delivery of the liquid and/or gas via one or more ejection port(s) 536 into the trocar channel 533. In some embodiments, the trocar 530 can include 1, 2, 3, 4, 5, 6, 7, 9, 10, or at least about 10 sensors 534. For example, in some embodiments, the trocar 530 can include a sensor 534 that can be configured to detect when an instrument (e.g., an imaging device) is close to the sensor (e.g., based on light detected by the sensor being above a predetermined threshold), and in response to detecting the light, the liquid and/or gas delivery can be triggered (e.g., via a controller). In some embodiments, the trocar 530 can include a first sensor that detects when an instrument (e.g., imaging device) is first inserted into the trocar channel 533 and a second sensor that detects when the instrument, having been previously inserted into the trocar channel 533, is being retracted for cleaning. In such embodiments, the first sensor, upon detecting that the instrument is being inserted into the trocar channel 533, can send a signal to a controller to not initiate a wash sequence as the instrument passes by the second sensor. Then with subsequent detection of the instrument by the second sensor (e.g., in response to a retraction of the instrument), the second sensor can send a signal to the controller to initiate the wash sequence. The sensor(s) 531 can include one or more light sensors, photoelectric sensors, pressure sensors, infrared sensors, force sensors, position sensors, piezoelectric sensors, mechanical sensors, etc.

The optional electronic port 535 can couple the sensor(s) 534 to the electrical line 582 and other electronic components of a cleaning system (e.g., controller 220). In some embodiments, the electronic port 535 is configured to provide power to the sensor(s) 534. In some embodiments, the electronic port 535 is configured to send to and/or receive data from the sensor(s) 534.

While the sensor(s) 534 and the electronic port 535 are described herein, it can be appreciated that the trocar 530 (and other trocars of cleaning systems described in the present disclosure) do not include any electronic components. In other words, the trocar 530 may not include the sensor(s) 534, the electronic port 535, or any other electronic components. Such omission of electronic components can reduce the complexity and therefore manufacturability and cost of the trocar 530. When used with a robotic system (e.g., robotic systems 180, 280, 380, etc.) as described herein, the trocar 530 need not include any electronic components, e.g., for detecting movement of an imaging device within the trocar channel 533 and determining when to initiate a wash sequence. Instead, the robotic system can be configured to determine when a wash sequence is needed and/or to position an imaging device for cleaning. In such cases, the robotic system can send a signal to a fluid delivery system (e.g., fluid delivery system 210) to cause the fluid delivery system to initiate the wash sequence. Additionally, the robotic system can position and/or orient the imaging device relative to the ejection port(s) 536 for suitable cleaning by the trocar 530.

The ejection port(s) 536 eject a gas and/or liquid (e.g., a wash solution) into the trocar channel 533, e.g., to wash a device such as, for example, an imaging device positioned in the trocar channel. In some embodiments, the ejection port(s) 536 can be angled retrograde or back towards a proximal end of the trocar 330 such that the ejection port(s) 536 eject the gas and/or liquid in a proximal direction, e.g., toward a distal end of an instrument. In some embodiments, the trocar 530 can include a single ejection port that is configured to generate a spray, e.g., for cleaning a distal end of an imaging device. In some embodiments, the trocar 530 can include a plurality of ejection ports for generating sprays. In some embodiments, the plurality of ejection portions can be set a different angles and/or orientations to cover a larger region within the trocar channel 533.

During use, a high pressure source of gas can be used to deliver a set volume of liquid (e.g., wash solution) into the trocar channel 533. The high pressure source of gas and the liquid can be coupled via the gas line 562 and the liquid line 572, respectively, to the liquid/gas interconnect 537. The liquid/gas interconnect 537 can combine the high pressure gas with the liquid, and with each wash sequence, allow the high pressure gas to draw and eject a set volume of liquid into the trocar channel 533. In some embodiments, the high pressure gas can be delivered at pressures of at least about 20 psi to at least about 50 psi, including all sub-ranges and values therebetween. For example, in an embodiment, the high pressure gas can be delivered at a pressure of at least about 30 psi, at least about 35 psi, or at least about 40 psi. Each wash sequence can last about 100 to about 500 ms, including all sub-ranges and values therebetween. For example, in an embodiment, the wash sequence can be at least about 100 ms to about 300 ms, including 200 ms.

The liquid being delivered by the ejection port(s) 536 can include water, a saline solution, a buffered solution, or a bio-compatible surfactant. For example, the liquid or wash solution can include a mixture of water and a surfactant. The mixture can include at least about 10% surfactant, about 15% surfactant, about 20% surfactant, about 25% surfactant, about 30% surfactant, about 35% surfactant, about 40% surfactant, or higher amounts of surfactant to water. During use with cleaning an imaging device, the distal end of the imaging device can be coated with a surfactant solution before being inserted into the trocar channel 533. The wash solution with a percentage of surfactant can then be used to wash the distal end of the imaging device, e.g., in one or more wash sequences when the imaging device is retracted. The presence of the surfactant in the wash solution can build a hydrophobic layer on the distal end of the imaging device, which can reduce fogging, water build-up, and other types of build-up on the distal end of the imaging device.

The vent 538 is fluidically coupled to the trocar channel 533. The vent 538 can be configured to allow for release for gases built up during surgery. In some embodiments, the vent 538 can be a passive vent, e.g., an opening that can allow gases to exit the body lumen and/or the cavity via the trocar channel 533 and vent 538. Alternatively, the vent 538 can be coupled to a vacuum source or other active component that can be used to regulate pressure within the body lumen and/or the cavity.

FIG. 6 provides detailed view of an obturator 690, according to an embodiment. As shown, the obturator 690 includes an obturator hub 692, an obturator shaft 694, and an optional obturator channel 693 extending the length of the obturator 690. As shown, the obturator 690 can be inserted into the trocar channel 633. In some embodiments, the trocar channel 633 can be the same or substantially similar to the trocar channel 533, as described above with reference to FIG. 5. Thus, certain aspects of the trocar channel 633 are not described in greater detail herein.

The obturator shaft 694 has sharp edges to facilitate initial placement of a trocar into the body lumen and/or cavity. For example, the distal end of the obturator shaft 694 can form a sharp, penetrating tip with a distal end of a trocar, and the penetrating tip can be used to cut through tissue while the trocar and obturator 690 are inserted through the tissue into the body lumen and/or cavity. In some embodiments, an imaging device can be placed in the obturator channel 693 during placement of the obturator 690 in the body lumen and/or cavity. In such embodiments, the imaging device can be positioned within the obturator channel 693 and used to capture image data of the patient anatomy as the obturator 690 is inserted into the body lumen and/or cavity. In some embodiments, the obturator 690 can be composed of a polymer, polyethylene, polypropylene, PVC, polycarbonate, polystyrene, or any other suitable material.

FIG. 7A depicts trocar 730 and obturator 790 of a cleaning system, according to embodiments. As seen in FIG. 7A, the trocar 730 (e.g., structurally and/or functionally similar to trocar 530 in FIG. 5) can include a trocar channel 733 (e.g., structurally and/or functionally similar to trocar channel 533 in FIG. 5), a trocar hub 731 (e.g., structurally and/or functionally similar to trocar hub 531 in FIG. 5), and an electronic port 735 (e.g., structurally and/or functionally similar to electronic port 535 in FIG. 5). The trocar 730 can also include a liquid/gas port 703 and/or a liquid/gas interconnect (e.g., structurally and/or functionally similar to the liquid/gas interconnect 537 in FIG. 5). The liquid/gas port 703 can be configured to receive a volume of liquid and/or gas, which can be delivered to an ejection port 736 at a distal end of the trocar 730. The ejection port 736 can be structurally and/or functionally similar to other ejection ports described herein, including, for example, ejection port 536 in FIG. 5.

FIG. 7B provides a more detailed view of the distal end of the trocar 730, showing ejection port 736 and one or more openings or transparent sections 717. The ejection port 736 can be configured to deliver one or more predetermined volumes or amounts of liquid and/or gas into the trocar channel 733. The ejection port 736 can be fluidically coupled to the liquid/gas port 703 via a passageway or channel 702. The openings or transparent section 717 can be configured to facilitate the use of one or more sensors. The trocar 730 can include one or more sensors (not depicted but similar to those described above with reference to FIG. 5), which can be used to detect a position of a distal end of an endoscope or other instrument within the trocar channel 733. In an embodiment, the one or more sensors can be light or optical sensors that are configured to detect when there is light within the trocar channel, e.g., as an indication of when a distal end of an endoscope is near the sensors. The one or more sensors can be positioned behind the openings or transparent sections 717 within a groove or channel 716. The groove or channel 716 can also house a wire, flexible circuit connection, and/or other electrical component 728 that operatively couples the one or more sensors to the electrical port 735. The electrical port 735 can be operatively coupled to a controller (controller 220, 320, etc.) of the cleaning system, e.g., for controlling the operation of the trocar 730 and/or delivery of liquid and/or gas, as further described below.

In use, an endoscope can be disposed within the trocar channel of the trocar 730, and one or more light sensors can be disposed at a location near an ejection port 736 of the trocar 730. In particular, at least one light source can be disposed at the same point along the longitudinal length of the trocar as the ejection port 736. The distal end of the endoscope can emit a light, e.g., for providing illumination within a body cavity for capturing image data. When the endoscope is withdrawn or retracted within the trocar channel, e.g., to initiate a wash sequence, the light emitted by the endoscope may be detected by the light sensor(s). In some embodiments, the light sensor(s) can be configured to detect when a level of light within the trocar channel is greater than a predetermined threshold or has changed by a predetermined amount or percentage. In response to there being sufficient light (e.g., the level of light being above the threshold), which can be indicative of the position of the distal end of the endoscope being in close proximity to the ejection port 736, the cleaning system (e.g., via a controller such as, for example, controller 220, 320, etc.) can cause a volume of liquid and/or gas to be ejected from the ejection port 736 to clean the distal end of the endoscope. In some embodiments, the light sensor(s) can provide sensor data indicative of the level of light within the trocar channel, and a controller (e.g., controller 220, 320, etc.) of the cleaning system can be configured to determine when that level of light is above a threshold for ejecting the liquid volume. Alternatively, the light sensor(s) can be triggered to send a signal to a controller of the cleaning system when the level of light within the trocar channel is above a threshold. While this example cleaning operation is provided herein with reference to light sensors, it can be appreciated that other types of sensor(s) can also be used with the trocar cleaning systems as described herein, including, for example, pressure sensors, motion sensors, etc.

The obturator 790 (e.g., structurally and/or functionally similar to obturator 690 in FIG. 6) can include an obturator hub 792 (e.g., structurally and/or functionally similar to obturator hub 692 in FIG. 6) and an obturator shaft 794 (e.g., structurally and/or functionally similar to obturator shaft 694 in FIG. 6). As such components have been described above with reference to FIGS. 1-6, details of such components are not described herein again.

Further suitable examples of trocars and/or obturators of cleaning systems are described in U.S. Provisional Patent Application No. 63/320,018, filed Mar. 15, 2022, titled “CLEANING DEVICES AND SYSTEMS FOR SURGICAL INSTRUMENTS INCLUDING GAS AND LIQUID DELIVERY AND VENTING,” and U.S. Provisional Patent Application No. 63/320,023, filed Mar. 15, 2022, titled “CLEANING DEVICES AND SYSTEMS FOR SURGICAL INSTRUMENTS INCLUDING TROCARS WITH SENSING AND FLUID DELIVERY,” the disclosure of each of which is hereby incorporated by reference in its entirety.

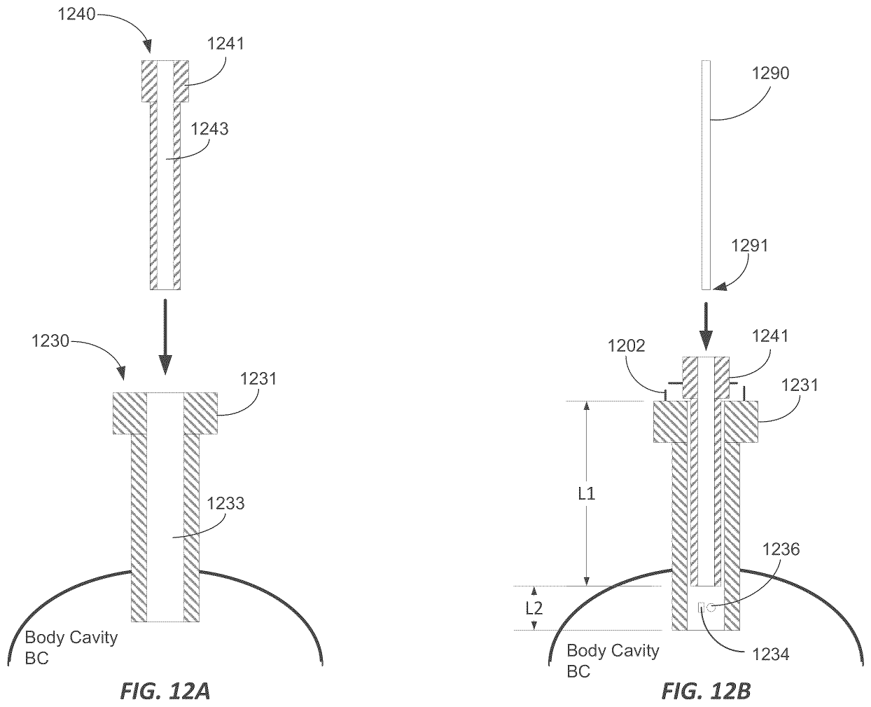

In some embodiments, a robotic system (not depicted) can be configured to control the insertion, positioning, and/or operation of the trocar 730. For example, the robotic system can be configured to couple to a portion of the trocar 730, e.g., the trocar hub. The trocar hub can include elements such as indentations, protrusions, slots, etc., e.g., for being gripped by a manipulator of a robotic system. In some embodiments, the robotic system can also be configured to attach to the obturator 790, e.g., via the obturator hub. The robotic system can be configured to insert the trocar 730 and the obturator 790 into a body lumen and/or cavity together, and then decouple the obturator and remove it from the trocar channel. Alternatively, in some embodiments, the trocar 730 and the obturator 790 can be inserted manually (e.g., by a surgeon) into a body lumen and/or cavity. Then a robotic system can optionally be coupled to the trocar 730 and/or be configured to insert an imaging device (e.g., an endoscope) into the trocar channel. The robotic system or the cleaning system (e.g., via controller 220, 320, etc.) can then subsequently control the washing of the imaging device. In some embodiments, the trocar 730 can be used with a trocar of a robotic system. For example, a trocar of a robotic system can be inserted into the trocar 730, and then an endoscope can be inserted through a channel of the trocar of the robotic system and placed within the body cavity. Further details of such an embodiment are described with reference to FIGS. 12A and 12B.

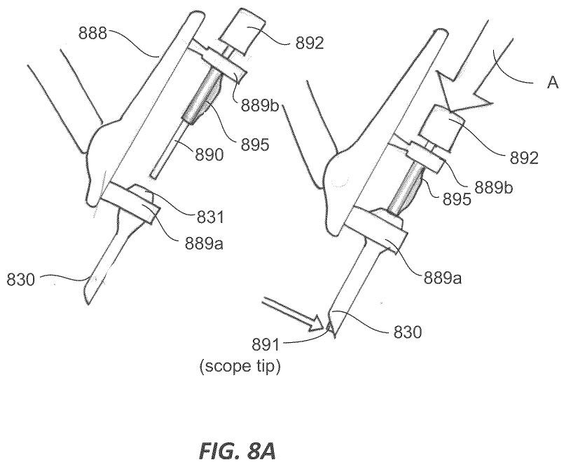

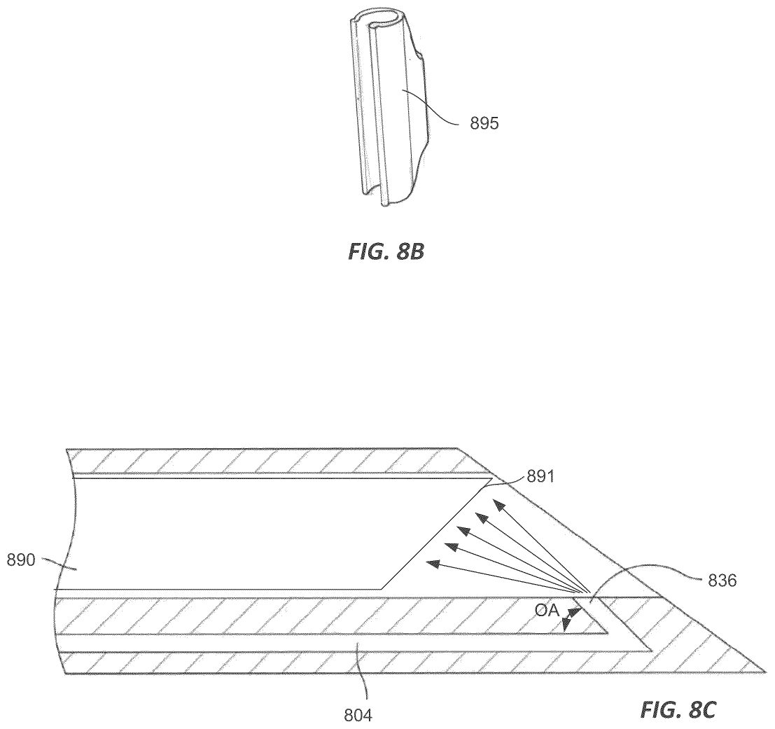

FIGS. 8A-8C depict the use of an adaptor 895 for calibrating the position and/or orientation of an imaging device 890 (e.g., structurally and/or functionally similar to imaging device 192 in FIG. 1) relative to a trocar 830 (e.g., structurally and/or functionally similar to trocar 130, 530, and 730). For instance, the adaptor 895 can be configured to calibrate the position and/or orientation of the imaging device 890 for a wash procedure using trocar 830. As seen in FIG. 8A, a robotic system can include a manipulator 888 (e.g., structurally and/or functionally similar to manipulator 486 in FIG. 4). The manipulator 888 can include two end effectors (e.g., structurally and/or functionally similar to end effector 489 in FIG. 4), such as end effector 889a and end effector 889b. The manipulator 888 can be coupled to the imaging device 890 via the end effector 889b. In a similar manner, the manipulator 888 can be coupled to the trocar 830 via the end effector 889a.

The adapter 895 can be configured to optimally position the imaging device 890 for a wash procedure. For example, adapter 895 can be sized such that imaging device 890 can be positioned at an optimal location relative to the trocar 830 (e.g., see FIG. 8C) for a wash procedure. More specifically, referring back to FIG. 8A, sizing the adapter 895 can cause the adapter 895 to set a distance between a proximal end 892 of the imaging device 890 and the proximal end (e.g., trocar hub) 831 of the trocar 830, such that the distal end 891 of the imaging device 890 can be optimally positioned for a wash procedure (e.g., positioned near an ejection port 836 of the trocar 830). For example, the end effector 889b can be configured to place the imaging device 890 in the trocar 830 such that the distal end 891 of the imaging device 890 is placed in a channel of the trocar 830. The end effector 889b and/or the end effector 889a can move the imaging device 890 and the trocar 830 respectively in a manner such that the imaging device 890 is inserted into the trocar 830 in the direction indicated by arrow A. The end effectors 889b and 889a can be configured to move the imaging device 890 and the trocar 830 until the adapter 895 prevents further movement of the imaging device 890 into the trocar 830. When the adapter 895 prevents further movement, the location of the imaging device 890 relative to the trocar 830 can be the optimal location of the wash procedure. In particular, when the adapter 895 prevent further movement, the distal end 891 of the imaging device 890 can be positioned proximal to an ejection port 836 of the trocar 830. In such position, liquid and/or gas that is delivered into the trocar, e.g., via a liquid/gas channel 804 and the ejection port 836, can be sprayed onto the distal end of the imaging device 890 for cleaning. In this manner, the imaging device 890 can be positioned at an optimal position relative to the trocar 830.

In some embodiments, the adapter 895 can also indicate an optimal orientation of positioning the imaging device 890 relative to the trocar 830, e.g., for when the imaging device 830 is an endoscope that has an angled distal end, as depicted in FIG. 8C. With an angled distal end, the endoscope may need to be positioned such that the angled surface of the endoscope faces the ejection port 836, e.g., for cleaning of the angled surface. Therefore, the endoscope may be rotated or orientated such that its distal surface 891 is positioned to face the ejection port 836. For example, the adapter 895 may include a ridge or other marking that can facilitate alignment or orientation of the endoscope relative to the ejection port 836 of the trocar 830. In particular, the ridge or marking can first be aligned with the side of the endoscope that extends further out, and then be aligned with a marking indicating the side of the trocar 830 opposite the ejection port 836. While the orientation of an angled endoscope is described herein, it can be appreciated that the orientation of the angled endoscope need not necessarily affect the cleaning of the endoscope. For example, sufficient pressures of liquid/gas spray may enable cleaning of the angled surface of the endoscope even when the angled surface of the endoscope does not face the ejection portion 836. For example, pressures of between about 20 psi and about 50 psi may be used to cause sufficient spraying of the liquid/gas into the trocar channel to clean the surface of any type of endoscope, including, for example, flat and angled endoscopes.

As shown in FIG. 8C, the orifice or ejection port 836 is angled in a proximal direction at an orifice angle OA. As described above, when the endoscope 803 has an angled distal end, it can be desirable to orient the endoscope such that the angled surface of the endoscope 803 faces the ejection port 836. This configuration allows for a substantial amount of the surface of the endoscope 803 to be contacted by the wash solution or liquid spray exiting the ejection port 836. In some embodiments, the orifice angle OA can be between about 5 degrees to about 90 degrees, inclusive of all values and sub-ranges therebetween.

In some embodiments, cleaning systems as described herein can be used to clean multiple different kinds and/or sizes of endoscopes. For example, the trocar 830 can have a trocar channel that is designed to receive endoscopes having an outer diameter of up to a predetermined value (e.g., up to about 10 French, about 15 French, about 20 French, about 30 French). Endoscopes then having diameters that fit within the trocar channel can be cleaned using the systems and methods described herein, regardless of the size and/or configuration of the endoscopes. For example, a trocar channel large enough to receive a 10 French endoscope can be configured to clean any endoscope that is smaller than or equal to 10 French, including for example, a 5 French endoscope. The cleaning systems can also be designed to clean endoscopes having different distal tip configurations, including, for example, different angles, curvature, etc. In some embodiments, to achieve this, the cleaning systems described herein can be configured to deliver a spray of liquid and/or gas at predetermined pressures above a threshold value that allows for sufficient cleaning of any shape or configuration of endoscope. In some embodiments, this threshold value can be between at least about 20 psi and about 50psi, including, for example, at least about 35 psi. Alternatively, the robotic systems described herein can be calibrated to position different imaging devices at different locations and/or orientations relative to a trocar of a cleaning system, e.g., for facilitating cleaning of those endoscopes.

In particular, although FIGS. 8A-8B illustrate an adapter with a circular cross-section, it should be readily understood that the adapter can be of any suitable shape and/or size that can position the imaging device 890 at an optimal distance relative to the trocar 830. For example, for long imaging devices (e.g., imaging devices with lengthy elongate body), the adapters 895 may be configured to be longer so as to position the imaging device 890 at an optimal distance. Therefore, adapters of different sizes and shapes may be configured for imaging devices of different sizes and/or shapes.

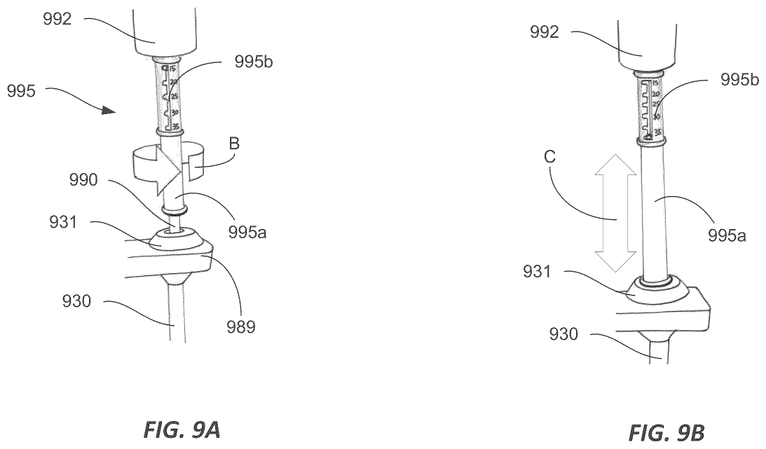

FIGS. 9A-9B depict another example of an adaptor 995 for positioning and/or orientating an imaging device 990 (e.g., structurally and/or functionally similar to imaging device 192 in FIG. 1) relative to a trocar 930 (e.g., structurally and/or functionally similar to trocar 130, 530, and 730), according to embodiments. In FIGS. 9A-9B, the adaptor 995 can include concentric tubes e.g., 905a, 905b, etc. These concentric tubes such as 905a and 905b can be positioned in a specific manner relative to one another so as to position the imaging device 990 at an optimal distance relative to the trocar 930. For example, the concentric tubes 905a, 905b can be locked at set distances from one another, e.g., to facilitate calibration/positioning of different imaging devices relative to the trocar 930. For example, the concentric tubes 905a, 905b can be rotated relative to one another to lock and unlock their positioning relative to each other (e.g., in a first direction as indicated by arrow B to unlock and in an opposite direction to lock). When locked, the concentric tubes 905a, 905b can define a set length, which can be associated with a set positioning of the distal end of an imaging device relative to the trocar 930, e.g., for conducting a wash sequence. For longer or shorter imaging devices and/or different types of imaging devices, the concentric tubes 905a, 905b can be set at different lengths, e.g., via translation as schematically illustrated via arrow C, to facilitate optimal positioning of those imaging devices. Therefore, adaptor 995 can be used to set different distances for imaging device 990 and trocar 930, which can be useful in different applications. For example, the same adaptor can be used for different types of imaging devices, different lengths of trocar, and/or different types of subjects. For instance, if the surgical procedure is to be performed in a child, the optimal distance of the imaging device would be different from the optimal distance if the same surgical procedure is to be performed in an adult.

In FIG. 9A, an imaging device 990 can be coupled to the adapter 995. The imaging device 990 can be inserted into the trocar 930 such that the distal end of the imaging device is inserted into the proximal end 931 of the trocar 930. When the imaging device 990 is at an optimal position and/or orientation relative to the trocar 930, the adaptor 995 can contact the hub 931 of the trocar 930, as shown in FIG. 9B.

In some embodiments, one or more of the tubes 995a and/or 995b can include markings on its outer surface. These markings can be indicative of a relative distance from the proximal portion of the adapter 995 to a distal tip of the imaging device 990, a relative distance from the distal portion of the adapter 995 to a distal tip of the imaging device 990, a reference point indicative of the optimal position for the imaging device 990, a total length of the adapter 995 that is exposed, a combination thereof, and/or the like.

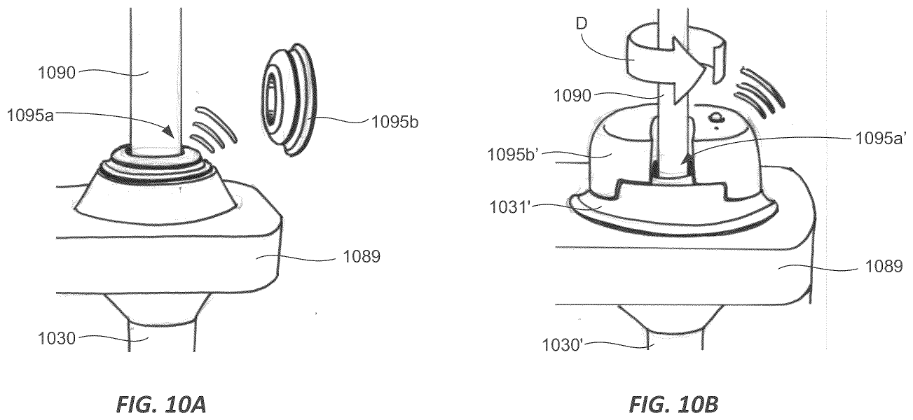

FIGS. 10A and 10B depict different types of external sensor systems 1095a, 1095b, 1095a′, 1095b′ that can be used to sense a position and/or orientation of an imaging device 1090 (e.g., structurally and/or functionally similar to imaging device 192 in FIG. 1) relative to a trocar 1030, 1030′ (e.g., structurally and/or functionally similar to trocar 130, 530, and 730), according to embodiments. In some embodiments, a tag (e.g., tag 1095a) can be placed on a portion of the imaging device 1090 (e.g., a base portion of the imaging device 1090) or on the manipulator of the robotic system (e.g., end effectors). The tag (e.g., tag 1095a) can be any suitable tag. For example, the tag can be a visual or fiducial marker (e.g., a visible marker such as a QR code, a barcode, etc.), a radio-frequency identification (RFID) tag, or the like. A sensor 1095b can be configured to sense a position of the tag 1095a. Therefore, as the imaging device 1090 moves (e.g., traverses) relative to the trocar 1030, the sensor 1095b can detect a position and/or orientation (or a change in position and/or orientation) of the portion of the imaging device 1090 (e.g., if the tag is on the imaging device 1090) and/or can detect a position and/or orientation (or a change in position and/or orientation) of the manipulator (e.g., if the tag is on the manipulator). The sensed data can be transmitted to the robotic system (e.g., processor 483 in FIG. 4). The compute device of the robotic system can analyze the position and/or orientation of the imaging device 1090 and/or the manipulator. The compute device can compare this position and/or orientation to the position and/or orientation of the trocar 1030 and/or a calibrated/optimal position of the imaging device for a wash sequence. In some embodiments, based on this comparison, the compute device can calculate the position and/or orientation of the imaging device 1090 relative to the trocar 1030. When the position and/or orientation of the imaging device (or the relative position and/or orientation of the imaging device) reaches an optimal position and/or orientation for cleaning, a wash procedure can be initiated to clean the imaging device 1090.

In FIG. 10B, a sensor 1095b′ can be positioned on a hub 1031′ of a trocar 1030′. The tag 1095a′ can be placed on the imaging device 1090. Accordingly, the sensor 1095b′ can sense the orientation of the imaging device 1090 relative to the trocar 1030′. The sensed information can be transmitted to the compute device in the robotic system. When the relative position and/or orientation reaches an optimal position and/or orientation, the robotic system can be configured to initiate the wash procedure to clean the imaging device 1090.

FIGS. 12A and 12B depict another example system for cleaning one or more instruments, such as, for example, an endoscope 1290, according to embodiments. As shown in FIGS. 12A and 12B, a trocar 1240 of a robotic system (e.g., a trocar coupled to a robotic arm or other manipulator (e.g., manipulator 486)) can be inserted or positioned within a trocar channel 1233 of a trocar 1230 of a cleaning system or fluid delivery system. The trocar 1230 can be structurally and/or functionally similar to other trocars of cleaning systems or fluid delivery systems described herein, including, for example, trocar 230, 330, 730, etc.

The trocar 1230 can include a trocar channel 1233, which can be sized to receive a trocar shaft of a trocar 1240 of a robotic system. The trocar 1240 can include a trocar channel 1243 that is sized to receive an endoscope 1290. As shown in FIGS. 12A and 12B, the trocar 1240 can be positioned within the trocar channel 1233, i.e., the trocar 1240 can be nested within the trocar 1230, and then the endoscope 1290 can be inserted into the body cavity BC through the trocar 1240. Similar to other trocars of cleaning systems described herein, the trocar 1230 can include a sensor 1234 and an ejection port 1236. In use, the sensor 1234 can be configured to sense when a distal end 1291 of the endoscope 1290 is positioned for cleaning (e.g., positioned near the ejection port 1236), and can send a signal to a controller of the cleaning system (e.g., controller 220, 320) to initiate a wash sequence.

In some embodiments, the trocar 1230 can include a hub 1231 that is designed to receive and/or mate with a hub 1241 of the trocar 1240. For example, the trocar hub 1231 can include an indented section, receptacle, etc. that can receive the hub 1241 of the trocar 1240. In some embodiments, the trocar hub 1231 and/or trocar hub 1241 can include magnets, adhesives, and/or other features for increasing engagement or hold between the trocar 1230 and the trocar 1240, e.g., to stabilize the trocars relative to one another during a surgical procedure. Optionally, in some embodiments, a coupler 1202 can be used to hold the trocar 1240 in the trocar 1230. The coupler 1202 can include, for example, a clamp, a tie, a sleeve, a wrap, a collar, a screw or other fastening element, or other like structure for holding the trocar 1240 in the trocar 1230.

The trocar 1240 can be received within the channel 1233 without extending to a distal end of the trocar 1230. In particular, the trocar 1230 can have a length that ensures that the sensor 1234 and the ejection port 1236 is distal to the distal end of the trocar 1240 when the trocar 1240 has been inserted a maximum length L1 into the channel 1233. The sensor 1234 and the ejection port 1236 can be located within a predetermined distance from a distal end of the trocar 1230 that is less than a distance L2 between the distal end of the trocar 1240 and the distal end of the trocar 1230 when the trocar 2140 has been inserted a maximum length L1 into the channel 1233. With the sensor 1234 and the ejection port 1236 remaining clear of the trocar 1240 (i.e., not being blocked by the trocar 1240), the sensor 1234 and the ejection port 1236 can be configured to perform cleaning of the endoscope 1290.

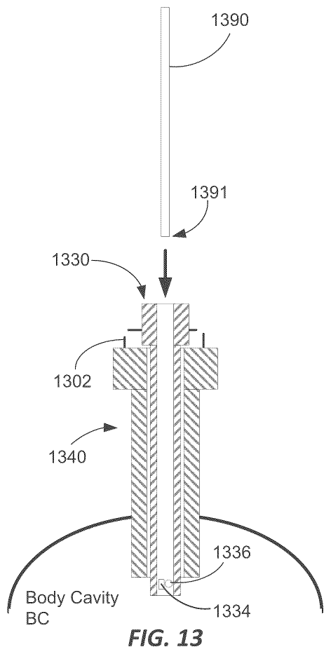

FIG. 13 depicts another example system for cleaning one or more instruments, such as, for example, an endoscope 1390, according to embodiments. As shown in FIG. 13, a trocar 1330 of a cleaning system or fluid delivery system can be inserted into a trocar 1240 of a robotic system (e.g., a trocar coupled to a robotic arm or other manipulator (e.g., manipulator 486)). The trocar 1330 can be structurally and/or functionally similar to the trocars of cleaning systems or fluid delivery systems described herein, including, for example, trocar 230, 330, 730, etc. For example, the trocar 1330 can include a sensor 1334 and an ejection port 1336 that can be configured to detect when the distal end 1391 of the endoscope 1390 is positioned near the ejection port 1336 for cleaning.

When the trocar 1330 of the cleaning system is positioned within the trocar 1340 of a robotic system, the trocar 1330 can be designed to extend distally from the trocar 1340. This can ensure or help reduce fluid or moisture build-up within the trocar 1340, e.g., as a result of wash sequences and/or cleaning. The fluids ejected by the trocar 1340 can exit into the body cavity BC.

Optionally, in some embodiments, a coupler 1302 can be used to hold the trocar 1330 in the trocar 1340. The coupler 1302 can include, for example, a clamp, a tie, a sleeve, a wrap, a collar, a screw or other fastening element, or other like structure for holding the trocar 1330 in the trocar 1340.

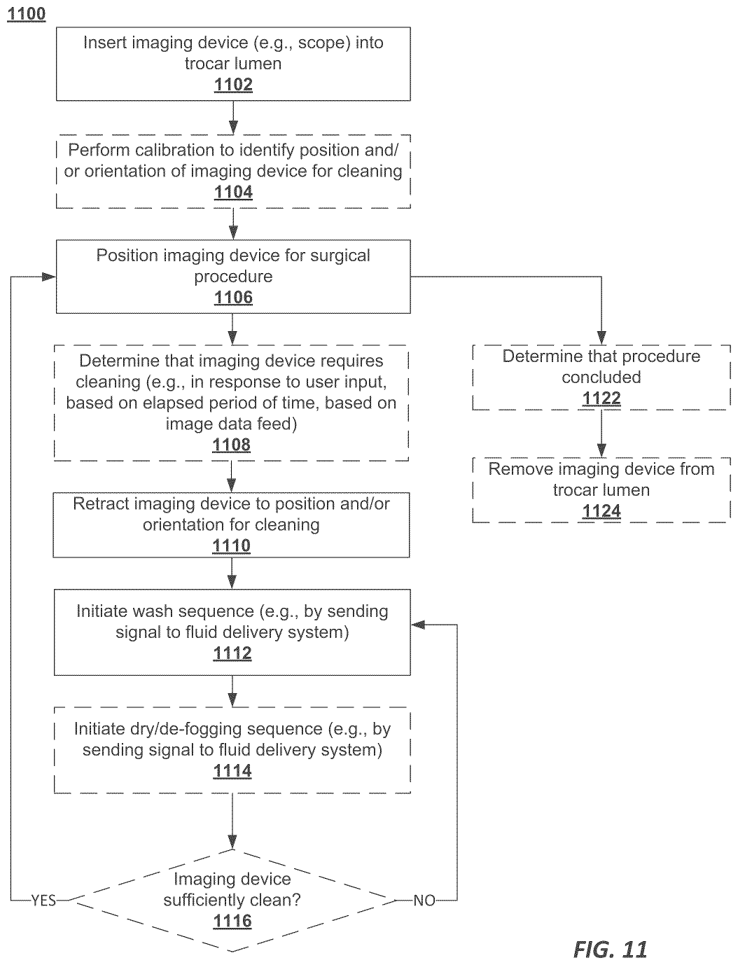

FIG. 11 depicts a flow of the operation 1100 of the robotic system (e.g., structurally and/or functionally similar to robotic system 180 in FIG. 1). At 1102, an imaging device can be inserted into a lumen of a trocar. In some embodiments, the robotic system can be configured to insert the imaging device into the trocar (e.g., via manipulator and end-effectors). Additionally or alternatively, a user can insert the imaging device into the trocar. At 1104, optionally, a calibration can be performed to identify position and/or orientation of the imaging device for cleaning. For example, an adaptor (e.g., adaptor 895 and 995) can be used to calibrate the position and/or orientation of the imaging device relative to the trocar such that the imaging device is at an optimal position for cleaning (e.g., the distal end of the imaging device is positioned proximal of an ejection port of the trocar).

After identifying the optimal position and/or orientation, the imaging device can be positioned (e.g., via manipulator and end-effectors) for the surgical procedure (e.g., at 1106). Optionally, at 1108, the operation can include determining that the imaging device requires cleaning. For example, a user can analyze images obtained from the imaging device. Based on the user's experience, the user can provide user input (e.g., via a user interface) indicating that the imaging device requires cleaning. Additionally or alternatively, the robotic system can analyze the image data from the imaging device. Based on this analysis, the robotic system can determine that the imaging device requires cleaning. For example, the robotic system can monitor the image data from the imaging device to determine when a level or intensity of light in the image data falls below a predefined threshold indicative of the imaging device being fouled. As another example, the robotic system can implement a trained machined learning model that can process the image data and provide an output indicative of whether the imaging device is fouled. Additionally or alternatively, the robotic system can determine time points at which the imaging device requires cleaning. For instance, the robotic system can determine if a predetermined amount of time from the start of the surgical procedure or after a cleaning sequence has elapsed. If the predetermined amount of time has elapsed, the robotic system can determine that the imaging device requires cleaning.

At 1110, the imaging device can be retracted to the optimal position and/or orientation for cleaning. For example, the imaging device can be retracted to an optimal position and/or orientation relative to the trocar, as determined at 1104. At 1112, a wash sequence can be initiated. For example, the robotic system can send a signal to the fluid delivery system to initiate a wash sequence. The controller of the fluid delivery system can initiate the wash sequence, e.g., by activating the pressurized gas to propel a predetermined volume of liquid into the trocar channel. Additionally or alternatively, the robotic system can directly interact with the fluid delivery system (e.g., via manipulator or end-effectors) to initiate the wash sequence. Additionally or alternatively, the controller of the fluid delivery system can receive signals from one or more sensors disposed in the trocar that indicate that the imaging device is positioned for cleaning (e.g., a distal end of the endoscope is positioned near an ejection port). In response to receiving the signal from the sensor, the controller can initiate the wash sequence. In such embodiments, the controller of the fluid delivery system and the robotic system can be separate devices and need not be operatively coupled to one another. This setup can allow for trocars of cleaning or fluid delivery systems as described herein to be used with many different types of robotic systems. In some embodiments, the wash sequence may involve propelling a predetermined volume of liquid using the pressurized gas and then delivering the pressurized gas during and/or following the ejection of the volume of liquid into the trocar channel.

At 1114, a dry/de-fogging sequence can be optionally initiated (e.g., by sending a signal to the fluid delivery system). During the dry/de-fogging sequence, pressurized gas may be ejected into the trocar channel at the imaging device, e.g., to dry the end of the imaging device. At 1116, the robotic system can optionally verify whether the imaging device is sufficiently clean. For example, the robotic system can obtain image data from the imaging device to verify whether the imaging device is sufficiently clean. If the imaging device is not sufficiently clean, the operation can loop back to 1112 to initiate the wash sequence again. If the imagining device is sufficiently clean, the robotic system can position the imaging device for surgical procedure again (e.g., at 1106). In some embodiments, the operation can include optionally determining that the surgical procedure is completed (e.g., at 1122) and removing the imaging device from the trocar lumen after the procedure is completed (e.g., at 1124).

While the examples provided herein are described with reference to cleaning imaging devices, it can be appreciated that other types of surgical devices can be cleaned using the systems, devices, and methods described herein. For example, surgical instruments other than endoscopes that are positioned within a trocar can also be cleaned using the systems, devices, and methods described herein.

Various concepts may be embodied as one or more methods, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments. Put differently, it is to be understood that such features may not necessarily be limited to a particular order of execution, but rather, any number of threads, processes, services, servers, and/or the like that may execute serially, asynchronously, concurrently, in parallel, simultaneously, synchronously, and/or the like in a manner consistent with the disclosure. As such, some of these features may be mutually contradictory, in that they cannot be simultaneously present in a single embodiment. Similarly, some features are applicable to one aspect of the innovations, and inapplicable to others.

As used herein, in particular embodiments, the terms “about” or “approximately” when preceding a numerical value indicates the value plus or minus a range of 10%. Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. That the upper and lower limits of these smaller ranges can independently be included in the smaller ranges is also encompassed within the disclosure, subject to any specifically excluded limit in the stated range. Where the stated range includes one or both of the limits, ranges excluding either or both of those included limits are also included in the disclosure.

The phrase “and/or,” as used herein in the specification and in the embodiments, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the embodiments, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the embodiments, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the embodiments, shall have its ordinary meaning as used in the field of patent law.

As used herein in the specification and in the embodiments, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the embodiments, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

As used in this specification, the singular forms “a,” “an,” and “the” include plural referents unless the context clearly dictates otherwise. Thus, for example, the term “a member” is intended to mean a single member or a combination of members, “a material” is intended to mean one or more materials, or a combination thereof.

The term “substantially” when used in connection with “cylindrical,” “linear,” and/or other geometric relationships is intended to convey that the structure so defined is nominally cylindrical, linear or the like. As one example, a portion of a support member that is described as being “substantially linear” is intended to convey that, although linearity of the portion is desirable, some non-linearity can occur in a “substantially linear” portion. Such non-linearity can result from manufacturing tolerances, or other practical considerations (such as, for example, the pressure or force applied to the support member). Thus, a geometric construction modified by the term “substantially” includes such geometric properties within a tolerance of plus or minus 5% of the stated geometric construction. For example, a “substantially linear” portion is a portion that defines an axis or center line that is within plus or minus 5% of being linear.

As used herein, the term “set” and “plurality” can refer to multiple features or a singular feature with multiple parts. For example, when referring to a set of devices, the set of devices can be considered as one device with multiple portions, or the set of devices can be considered as multiple, distinct devices. Thus, a set of portions or a plurality of portions may include multiple portions that are either continuous or discontinuous from each other. A plurality of particles or a plurality of materials can also be fabricated from multiple items that are produced separately and are later joined together (e.g., via mixing, an adhesive, or any suitable method).

While specific embodiments of the present disclosure have been outlined above, many alternatives, modifications, and variations will be apparent to those skilled in the art. Accordingly, the embodiments set forth herein are intended to be illustrative, not limiting. Various changes may be made without departing from the spirit and scope of the disclosure. Where methods and steps described above indicate certain events occurring in a certain order, those of ordinary skill in the art having the benefit of this disclosure would recognize that the ordering of certain steps may be modified and such modification are in accordance with the variations of the invention. Additionally, certain of the steps may be performed concurrently in a parallel process when possible, as well as performed sequentially as described above. The embodiments have been particularly shown and described, but it will be understood that various changes in form and details may be made.

1. An apparatus, comprising:
a shaft having a distal end that is disposable within a subject's anatomy, the shaft including an ejection port disposed near the distal end of the shaft, the ejection port configured to eject a volume of liquid or gas into the channel to clean a distal end of the instrument;
a hub coupled to a proximal end of the shaft, the hub and the shaft collectively defining a channel for receiving a trocar of a robotic system;
a sensor disposed near the ejection port, the sensor configured to detect when a distal end of an instrument disposed within a channel of the trocar is disposed near the ejection port; and
a controller operatively coupled to the sensor, the controller configured to:
receive, from the sensor, a signal indicative of the distal end of the instrument being disposed near the ejection port; and
in response to receiving the signal from the sensor, activate a wash sequence by triggering the ejection of the volume of liquid or gas.

2. The apparatus of claim 1, wherein the hub includes an indentation or receptacle for receiving at least a portion of a hub of the trocar of the robotic system.

3. The apparatus of claim 1, further comprising one or more coupling elements configured to couple the trocar of the robotic system to the shaft when the trocar is received in the channel.

4. The apparatus of claim 3, wherein the one or more coupling elements includes a clamp, a tie, or a collar.

5. The apparatus of claim 3, wherein the one or more coupling elements includes at least one magnet or an adhesive.

6. The apparatus of claim 1, wherein the shaft has a length such that, when the trocar is fully received in the channel, a distal end of the trocar is proximal of a distal end of the shaft.

7. The apparatus of claim 6, wherein the ejection port and the sensor are disposed distal to the distal end of the trocar when the trocar is fully received in the channel.

8. A system, comprising:
a shaft defining a channel for receiving an instrument, the shaft having a distal end that is disposable within a subject's anatomy, the shaft including an ejection port configured to eject a volume of liquid or gas into the channel to clean a distal end of the instrument;
a fluid delivery system coupled to the shaft and configured to control the ejection of the volume of liquid or gas;
a robotic system coupleable to the instrument and configured to retract the instrument to a predetermined position; and
a processor operatively coupled to the robotic system and the fluid delivery system, the processor configured to control the fluid delivery system to cause the ejection of the volume of liquid or gas in response to the instrument being retracted to the predetermined position.

9. The system of claim 8, wherein the robotic system includes the processor.

10. The system of claim 8, wherein the instrument is an endoscope, and the processor is further configured to monitor image data captured by the endoscope to detect when a lens of the endoscope requires cleaning,
the processor configured to cause the robotic system to retract the endoscope to the predetermined position in response to detecting that the lens of the endoscope requires cleaning.

11. The system of claim 10, wherein the processor is configured to detect that the lens of the endoscope requires cleaning based on an intensity of light in the image data captured by the endoscope.

12. The system of claim 8, wherein the predetermined position is when a distal end of the instrument is positioned near the ejection port.

13. The system of claim 8, further comprising:
an adapter configured to couple to the instrument, the adapter configured to be used to indicate to the robotic system a length to retract the instrument to place the instrument in the predetermined position.

14. The system of claim 8, further comprising:
a sensor disposed on at least one of the instrument or the trocar, the sensor configured to detect when the instrument has been retracted to the predetermined position.

15. The system of claim 14, wherein the sensor is configured to detect a position of a tag to detect when the instrument has been retracted to the predetermined position.

16. A method, comprising:
positioning a trocar within a shaft of a cleaning system such that a distal end of the trocar is disposed within a channel of the shaft, the shaft having a proximal end that is coupled to a hub and a distal end that is disposed within a body cavity;
securing a proximal end of the trocar to the hub after the trocar has been positioned within the shaft;
inserting an endoscope into a channel of the trocar such that a distal end of the endoscope extends distally from the distal end of the trocar, into the channel of the shaft, and then into the body cavity; and
retracting the endoscope such that the distal end of the endoscope is within the channel of the shaft and near an ejection port within the shaft to be cleaned by a volume of liquid and gas that is ejected by the ejection port into the channel.

17. A method, comprising:
detecting that a distal end of an endoscope is near an ejection port of a first trocar disposed in a body cavity, the endoscope being supported within a channel of a second trocar that is disposed within a channel of the first trocar;
in response to the detecting, activating a delivery of pressurized gas to propel and deliver a predetermined volume of liquid to the first trocar; and
delivering, via the ejection port, the pressurized gas and the predetermined volume of liquid into the channel of the first trocar to clean the distal end of the endoscope.
